SEQUENCE LISTING (32 sequences)

SEQ ID NO: 1 - protein, 1166 aa, Infectious bronchitis virus
MSVTRLLLVTLLFALCSAVLYDSNSYVYYYQSAFRPGSGWHLHGGAYAVVNVSEQTSPGSCTAGAIGYSMNFSAASVAMTAPLNGMSWSDRQFCMAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTCVLHNFTFFNETSAPPAGGDISARFPTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTDSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEYIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELLKWWNETKHDLPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 2 - protein, 1079 aa, Infectious bronchitis virus
VLYDSNSYVYYYQSAFRPGSGWHLHGGAYAVVNVSEQTSPGSCTAGAIGYSMNFSAASVAMTAPLNGMSWSDRQFCMAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTCVLHNFTFFNETSAPPAGGDISARFPTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTDSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEYIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELLKWWNETKHDLPDFDQFNYTIPVLNITYDIDKIEEVIKGLNDSLIDLETLSILKTYIKWP

SEQ ID NO: 3 - DNA, 27646 bp, Infectious bronchitis virus
  source 1..27646: mol_type = genomic DNA
actaaaaatagatattaatatatatctattacactagccttgcgctagatttccaacttaacaaaacggacttaaatacctacagctggtcctcataggtgttccattgcagtgcactttagtgccctggatggcacctggccacctgtcaggtttttgttattaaaatcttattgttgctggtatcactgcttgttttgccgtgtctcactttatacatccgttgcttgggctacctagtatccagcgtcctacgggcgccgtggctggttcgagtgcgaagaacctctggttcatctagcggtaggcgggtgtgtggaagtagcgcttcagacgtactggttctgttgcgtgaaacgcggggtcacctccccccacatacctctaagggcttttgagcctagcgttgggctacgttctcgcacaaggtcggctatacgacgtttgtagggggtagtgccaaacaacccctgaggtgacaggttctggtggtgtttagtgagcagacatacaatagacagtgacaacatggcttcaagcctaaaacagggagtatctcccaaaccaagggatgtcattcttgtttccaaagacattcccgaacaactctgtgacgctttatttttctacacgtcacacaaccctaaggattacgctgatgcttttgcatttaggcaaaagtttgaccgtaatctgcagactgggaagcagttcaaatttgaaactgtttgtggtctcttcctattgaagggagttgacaaaataacacctggcgtcccagcaaaagttttaaaagccacttctaagttggcagatttagaagacatctttggtgtctctccttttgcacggaagtaccgtgaattgttgaaaacagcatgccagtggtctcttactgtagaaacactggatgctcgtgcacaaacgcttgacgaaatttttgactctactgaaatactttggcttcaggtggctgcaaaaattcaagtttcagctatggcaatgcgcaggcttgttggagaagtaactgcaaaagtcatggaagctcttggctcaaatttgagtgttctctttcaaattgttaaacaacaaatagccagaatctttcaaaaggcactggctatttttgaaaatgtgagtgaattaccacagcgtattgcagcacttaagatggcctttgccaagtgtgccaagtcaattactgttgtggttgtggaaagaactctagttgttagagagttcgcaggaacttgtcttgcaagcatcaatggtgctgttgcaaaattctttgaagaacttccaaatggctttatgggttctaaaatcttcacaacattggccttctttaaagaagcagctgtgaaaattgtggaaaatataccaaatgcaccaagaggtactagaggttttgaagtcgttggtaacgccaagggaacgcaagttgttgtgcgtggcatgcgaaatgatttaactctgctcgaccaaaaagctgacattcctgttgagaaagaaggttggtctgcaattcttgaaggacatctgtgttatgtctttaagagtggtgatcgtttttatgcggcacctctttcagggaattttgcattgcatgatgtgcattgttgtgagcgtgttgtctgtctgtctgatggtgtaacaccagagataaatgatggactcattctagcagcaatctattcatcttttagtgtctcagaactcgtggcagcacttaaaaagggtgaaccattcaagttcttgggtcataaatttgtgtatgcgaaggatgcagcagtctctttcactcttgcaaaagcagccactattgcagatgtactgaagctgtttcaatcagctcgtgtgcaaacagaagatgtgtggtctgcatttactgaaaagtcttttaatttctggaaactcgcatatggaaaagtgcgtaatcttgaagaagttgtgaagactcatttttgtaaagctcaaatgtcaattatcattctagcagcagtgcttggcgaaggcatttggcatcttgtttcacaggtcatctataaagtaggtggtctttttactagagtcgttgacttttgtgaaaaacactggaagggtttctgtgcacaacttaaaaaggctaagctcgttgtcacagaaactctttgtgttcttaagggagtggcacagcattgttttcaactattgctggatgcaatacattctttgtatatgagttttaagaagtgtgcacttggtagaattcatggagacttactcttctggaaagggggtgtacacaaaattgttcaagatggcgatgaagtttggtttgacgccattgatagtattgatgttgaagatctgggtgttgtccaagaaaaacccatagattttgaggtttgtgaagacgtaacacttccagaaaatcaacctggtcatatggttcaaatcgaggatgacggaaagaactatatgttcttccgcttcaaaagggatgagaacatctactatacaccaatgtctcaacttggtgcaattaatgtagtttgcaaagcaggcggtaaaaccgttacctttggagacaccattgtgaaagaaataccgccacctgatgttgtgcctattaaggttagcatagagtgttgtggtgaaccatggaatacaatcttcaagaaagcttataaagagcccattgaagttgaaactgacctcacagtagaacaattgctctctgtgatctatgagaaaatgtgcgacgacctcaaattgtttccagaggcaccagagccaccgccatttgagaatgtcgcacttgttgataaaaacggtaaagacttggattgcataaaatcatgccatcttatctaccgtgattatgagagtgatgatgacatcgaggaagaagatgctgaggagtgtgatactgatttagaatgtgaagaagaggatgaggatactaaagtgttggctcttatacaagaccctgcaagtaataaataccctcttcctcttgatgatgattatagcgtctttaatggatgtattgtacataaggacgctcttgacgtcgtaaatctaccatctggtgaagaaacctttgttgtcaacaactgctttgagggagctgtaaaaccactgcctcagaaagttgttgatgttctaggtgactggggtgaggctgttgatgcgcaagagcaaattgcacaaactacttcagaggaaacccctatcagtagtttggaggcaactattgagcaagttgttgttgaggaacagaaaataatttctgttgttgaagaagaacagcaggtggcggtctacacacctgcagacctacaagttgttgaagaaacaccagatgagtttattcttattgctgatgtttccacagaagaaattgtgcctcatgaagaaaaggagtcacagattgaacaggagcctattcaagttgttaaatcacaacgtgaaaagaaggctaaaaagttcaaggttaaatctactacatgtgagaaacccaaatttttggagtacacaacatgtgtgggtgacctaacggtagtgattgccaaagcattggatgagtttaaagagttctgcattgtaaatgctgctaatgagcatatgtctcacggtggcggcgttgcaaaggcaattgcagacttttgtggaccggactttgttgaatattgcgcggactatgttaagaaacatggtccacagcaaaaacttgtcacaccttcatttgttaaaggcattcaatgtgtgaataatgttgtaggacctcgccatggagacagcaacttgcgtgagaagcttgttgctgcttacaagagtgttcttgtaggtggagtggttaactatgttgtgccagttctctcatcagggatttttggtgtagattttaaaatgtcaatagatgctatgcgcgaagcttttaaaggttgtgccatacgcgttcttttattttctctgagtcaagaacacatcgattatttcgatgcaacttgtaagcagaagacaatttatcttacggaggatggtgttaaataccgctctgttgttttaaaacctggtgattctttgggtcaatttggacaggtttttgcaagaaataaggtagtcttttcggctgatgatgttgaggataaagaaatcctctttatacccacaactgacaagactattcttgaatattatggtttagatgcgcaaaagtatgtaatatatttgcaaacgcttgcgcagaaatgggatgttcaatatagagacaattttgttatattagagtggcgtgacggaaattgctggattagttcagcaatagtgctccttcaagctgctaagattaggtttaaaggttttcttgcagaagcatgggcacaacttttgggtggagacccaactgattttgtagcctggtgctatgcaagttgcaatgctaatgttggtgagttttcagatgctaattggcttcttgctaatttggcagaatactttgatgctgattacacgaatgcattccttaagaggcgtgtgtcatgtaactgtggggttaagaattgtgaagttagaggccttgaagcttgtattcaaccagtaaaggcacccaatcttcttcatttcaagactcagtacacaaattgtacagtgtgtgatgcaaatagtgtggatgaggtggtagaggcctcactaccatatctgttgctccttgctactgatggtcctactacagtggattgtgatgaaaatgctgtagggaatgttgttttcattggctctactaatagtggccattgttacacgcaagccattggtaaggcttttgataatcttgctaaggatagaaaattttcaaagaattcgccatacattacagcaatgtatacgcgcttctctcttaagagtgaaagctctctgtctgttgttaaacagagtaagagtaaaactaaagtagtaaaagaagatgttgccaaccttgttactagttctaaagccagttttgatgatcttactgactttgaacattggtatgatagtaacatctatgaaagtcttaaagttcaggaaatacctgtgaatttggatgagtatgtgtcatttacaacgaaagaagatactaagttgccactgacacttaaagttagaggtatcaaatcagttgttgactttatttcaagagacggtttctcttataagttaacacctgacattgaagaaaattcaaaagcgccagtctactacccagtcttagactctattagtcttaaggcaatatgggtagacggcagtgctaattttgttgttggtcatccaaactactatagtaagtctcttcgcattcctactttttgggaaaatgcagagagctttgttaagataggtgacaaagttgatggtgtaactatgggcctttggcgtgcagaacagcttaacaaacctaatcttgaaagaattttcaacattgctaagaaagctattgttggatccagtgttgttactacacaatgtagtaaattaattagtaaagcagctacattcattgctgataaagtaggtgggggtgtagttcgtaatattacagatagaattaagggtctttgtggatttacacgtgggcattttgaaagaaaattgtctccacaattcataaaaacacttatattcttcttcttttactttgtaaaggctagtgctaagagtgttgccactagttataagcgtgtgttatgtaaggtggtttttaccacgctatttatattatggtttatgtacacaagcaaaccagtaacttttactggaacacgtgtgctagacttcttatttgagggttctttatgtggtccctataatgactatggtaaagactcatttgacgtactacgctattgtggagatgattttacttgtcgtgtatgtttacatgataaagattcacttcatttgtataagcatgcttatagcgtagaacaggtttataaagatgcagcttctggcattagttttaattggaattggctttatttggtctttctaatattatttgttaaaccagtagcaggtttcgttattatttgctattgtgttaagtacttggtattgagttcaactgtgttgcaaactggtgtaggttttatggactggtttattcaaacagtttttactcactttaattttatgggtgcaggtttctatttctggctcttttataaattgtacatacaggttcatcatatactgtattgtaaggatataacatgtgaagtgtgtaagagagttgcacgcagtaacaggcatgaggttagtgttgttgttggtggacgcaagcaaattgtgcatgtgtacactaactctggttacaacttttgtaagagacataattggtattgtaggaattgtgatgtatatggtcaccaaaacacatttatgtctcctgaagttgctggcgagctttctgaaaagcttaaacgccatgttaaacctacagcacatgcttaccacgttgtggatgaggcttgcgtagttgatgattttgttaacttaaaatacaaagctgcaactcctggtaaggatggtgcacctcctgcagttaaatgtttcagtgttacagatttcttgaagaaagctgtttttcttaaggatgcactgaaatgtgaacaaatatctaatgatggttttatagtgtgtaatacgcagagtgcgcatgctttagaggaagcaaagaatgcagccatctattatgcgcaatacctgtgtaaacctatacttatactcgaccaggcactctaccagaatttaatagtggaacctgtatcgaagagcgttgtcaacaaagtgtgtgacattttgtctaggataatttctgtagatactgcatctttggattataaagcaggtacaattcgtgatgccttgctgtctgttactaaagatgaagaagctgtagatatggctatcttctgtcataatcatgaagttgaatatacaggtgatggttttactaatgttataccgtcatatggtatagacactgataaattaacacctcgtgatagagggtttttgataaatgcagatgcttctgttgctaacttaagagttaaaaatgctccgccggtagtatggaagttctctgatcttattaagttgtctgacagttgtcttaaatatttaatctcagcaactgtcaagtcagggtctcgtttctttataacaagatctggtgctaaacaaattttttcttgtagtactcagaaattgttggtagagaaaaaggctggtggtgtcattagtggtacctttaattggtttaagagttgttgtaaatggctcttgatcttctatgtgctttttacattgtgttgtttgggttgttatcatatggagacgaataaaagttttgttcatcctatgtatgatgttaactctacaatgcatgttgaaggctttaaggttatagataaaggtgttattagagacattgtaccagaggatgcttgtttctctaataagtttgctaactttgatgcattttggggtaaaccatatgtgaatagtagagactgtccaattgttacagcagtcatagatggcgctggaacaatagcagctggtgttcctggttttgtagactgggttcttgatggtgttatgtttgtacacatgacacaaacagaaagaaaaccctggtacattcccacgtggtttaacagagaaattgttggttacactcaggattcaattattactgaaggtagtttttatacatctatagctttgttttcagctaggtgtttatatttaacagccagcaatacaccacaattgtattgttttaatggtcataatgatgctcctggagccttaccatttagcagtatcactccacacagggtctacttccaaccaaatggtgttaggcttataattcctcaacagataatgcacacaccctacgtagtaaagtttttatcagacagctattgtagaggtagtgtatgtgagtatactaaaccgggttattgtgtctcactaaattcccaatgggttttatttaatgacgaatacacaagtaaaccaggagtattctgtggttctactgttagagaacttatgtttaatatggttagtacattttttactggtgtcaaccctaatatttatatgcagctggcgactatgttcttaatactagttgttgttgtgttaatttttgcaatggttataaagtttcaaggtgtttttaaagcttatgcaaccattgtgtttacaataatgctagtttgggttgttaatgcatttattttgtgtgtacatagttataatagtgttgtggctgttatactactagtaatctattgttatgcatcattggttacaagtcgtaatactgctataataatgcattgttggcttgtgtttacctttggtttaattgtacccatatggttggcgtgttgctacctggcatttgttttatatatgtacacaccattgtttttctggtgttacggtactactaaaaacactcgtaaattgtatgatggcaacgagtttgttggtacttatgatcttgctgcgaagagcacctttgttattcgcggtcctgaatttgttaagcttacgaacgagataggtgataagtttgaacactatctctcagcgtatgctagacttaaatactactcaggcactggcagtgaacaagattatctgcaagcttgtcgtgcatggttagcttatgctttggaccaatatagaaatagtggtgtggaaattgtgtatactccaccacgttactctattggtgttagtagattacaggccggttttaagaaactagtttctcctagtagtgttgttgaaaagtgcattgttagtgtctcttatagaggtaataatcttaatggactgtggctaggtggtactatctactgtccgcgacatgttctaggcaagttctcaggtgaccaatggaatgatgtacttaaccttgctaataatcatgagtttgaagttgtaactcaaaataatgttactttgaatgttgtcagtaggaggctaaaaggtgcagttttgattttacaaactgctgttgctaatgctgaaactccaaagtataagtttgttaaagccaattgtggagatagttttacgattgcttgttcctatggtggtacagttgttggactctaccctgttactatgcgctctaatggtactattagggcgtctttcttagcaggagcgtgtggttctccaggttttaatatagaaaagggtgtagttaacttttattatatgcaccatcttgagttgcctaatgcattacacacaggaactgacctaatgggagagttctatggtggttatgtggacgaagaggttgcacaaagggtgccaccagataatttagttactaacaatattgtagcatggctttatgccgcaattattagtgttaaggagagtagtttctcactgcctaaatggttggatagtactactgtcagtgttgaagactgcaataagtgggctggtgataatggttttacaccattttctactagtactgctattactaaattaagtgctataacaggagtagatgtttgtaaactccttcgcactattatggtaaaaagtagtcaatggggtagtgatcccattttaggacaatataattttgaagatgaattgacaccagagtccgtttttaatcagataggtggtgttagattacagtcttcttttgtaagaagagccacatcctggttttggagtagatgtgtgttagcttgcttcttatttgtgttgtgtgctattgtcttgtttacggcagtgccacttaaatattatgtacatgcagccgttattttgttaacagctgtgctcttcatttcttttactgttaaacatgttatggcatatatggatacttttctactgccaacattgcttacagtcattattggagtttgtgctgaagtacctttcatctacaatactctaattagtaggatagttgtctttgttagtcaatggtatgatcctgtagtctttgatactatggtaccatggatgttcttgccactagtgttgtacacagcatttaagtgtgtgcagggttgctatagtgtgaattctttcaatacttctttgctagtactgtaccagttcttgaagttaggctttgttatttatgcctcttctagcacgctggcagcatacacagaaggtaattgggatttattttttgaattagttcacactactgtgttggctaatgttagtagtaattccttaataggtttgtttgtgttcaagttagctaagtggatgttgtattattgtaatgctacatactttaataattatgtgctaatggctgtcattattaatggctttggttggctcttcacttgttactttggagtttattggtggattaataaggtttttggtttaaccttaggtaaatatgaattcaaagtttcagtagaccaatataggtatatgtgtcttcataagataaattcgcctaaaactgtgtgggaagttttttcgacaaatatacttatacaaggaattggtggtgatcgtgtgttgcctatagctacagtgcaatctaaattgagtgatgtaaagtgtacaactgttgttttaatgcagcttttgactaagcttaatgttgaagcaaattcaaaaatgcatgcttatcttgttgagttacacaataaaatcctcgcatctgatgatgttggagagtgcatggataatttattgggtatgcttataacactattttgtatagattctactattgatttgggtgagtattgtgatgatatacttaagaggtcaactgtattacaatcggttactcaagagttttcgcacataccctcgtatgctgaatatgaaagagctaagagtatttatgaaaaggttttagccgattctaaaaatggtggtgtaacacagcaagagcttgctgcatatcgtaaagctgccaatattgcaaagtcagtttttgatagagacttggctgttcaaaagaagttagatagcatggcagaacgtgctatgacaacaatgtataaagaggcgcgtgtaactgatagaagagcaaaattagtttcatcattacatgcactacttttttcaatgcttaagaaaatagattctgagaagcttaatgtcttatttgaccaggcgaatagtggtgttgtacccctagcaactgttccaattgtctgtagtaataagcttaccctcgttataccagacccagagacgtgggtcaagtgtgtggagggtgtgcatgttacatattcaacagttgtttggaatatagactgtgttactgatgccgatggcacagagttacaccccacttctacaggtagtggattgacttactgtataagtggtgataatatagcatggcctttaaaggttaacttgactaggaatgggcataataaggttgatgttgccttgcaaaataatgagcttatgcctcacggtgtaaagacaaaggcttgcgtagcaggtgtagatcaagcacattgtagcgttgagtctaaatgttattatacaagtattagtggcagttcagttgtagctgctattacctcttcaaatccaaatctgaaagtagcctcttttttgaatgaggcaggtaatcagatttatgtagacttagacccaccatgtaaatttggtatgaaagtgggtgataaggttgaagttgtttacctgtattttataaaaaatacgaggtctattgtaagaggtatggtacttggtgctatatctaatgttgttgtgttacaatctaaaggtcatgagacagaggaagtggatgctgtaggcattctctcactttgttcttttgcagtagatcctgcggatacatattgtaaatatgtggcagcaggtaatcaacctttaggtaactgtgttaaaatgttgacagtacataatggtagtggttttgcaataacatcaaagccaagtccaactccggatcaggattcttatggaggagcttctgtgtgtctttattgtagagcacatatagcacaccctggcggagcaggaaatttagatggacgctgtcaatttaaaggttcttttgtgcaaatacctactacggagaaagatcctgttggattctgtctacgtaacaaggtttgcactgtttgtcagtgttggattggttatggatgtcagtgtgattcacttagacaacctaaaccttctgttcagtcagttgctgttgcatctggttttgataagaattatttaaacgggtacggggtagcagtgaggctcggctgataccccttgctaatggatgtgaccccgatgttgtaaagcgagcctttgatgtttgtaataaggaatcagccggtatgtttcaaaatttgaagcgtaactgtgcacgattccaagaagtacgtgatactgaagatggaaatcttgagtattgtgattcttattttgtggttaaacaaaccactcctagtaattatgaacatgagaaagcttgttatgaagacttaaagtcagaagtaacagctgatcatgatttctttgtgttcaataagaacatttataatattagtaggcagaggcttactaagtatactatgatggatttttgctatgctttgcggcactttgacccaaaggattgcgaagttcttaaagaaatacttgtcacttatggttgtatagaagattatcaccctaagtggtttgaagagaataaggattggtacgacccaatagaaaaccctaaatattatgccatgttggctaaaatgggacctattgtacgacgtgctttattgaatgctattgagttcggaaacctcatggttgaaaaaggttatgttggtgttattacacttgataaccaagatcttaatggcaaattttatgattttggtgattttcagaagacagcgcctggtgctggtgttcctgtttttgatacgtattattcttacatgatgcccatcatagccatgactgatgcgttggcacctgagaggtattttgaatatgatgtgcataagggttataaatcttatgatctcctcaagtatgattatactgaggagaaacaagagttgtttcagaagtactttaagtattgggatcaagagtatcaccctaactgtcgcgactgtagtgatgacaggtgtttgatacattgtgcaaacttcaacatcttgttttctacacttgtaccgcagacttctttcggtaatttgtgtagaaaggtttttgttgatggtgtaccatttatagctacttgtggctatcattctaaggaacttggtgttattatgaatcaagataacaccatgtcattttcaaaaatgggtttgagtcaactcatgcagtttgttggagatcctgccttgttagtggggacatccaataaattagtggatcttagaacgtcttgttttagtgtttgtgctttagcgtctggtattactcatcaaacggtaaaaccaggtcactttaacaaggatttctacgattttgcagagaaggctggtatgtttaaggaaggttcttctataccacttaaacatttcttctacccacagactggtaatgctgctataaacgattatgattattatcgttataacaggcctaccatgtttgatatacgtcaacttctattttgtttagaagtgacttctaaatattttgaatgttatgaaggcggctgtataccagcaagccaagttgtagttaacaatttagataagagtgcaggttatccgttcaataagtttggaaaggcccgtctctattatgaaatgagtctagaggagcaggaccaactctttgagagtacaaagaagaacgtcctgcctactataactcagatgaatttaaaatatgccatatccgcgaaaaatagagcgcgtacagtggcaggtgtgtctatcctttctactatgactaataggcagtttcatcagaagattcttaagtctatagtcaacactagaaacgctcctgtagttattggaacaaccaagttttatggcggttgggataacatgttgagaaaccttattcagggtgttgaagacccgattcttatgggttgggattatccaaagtgtgatagagcaatgcctaatttgttgcgtatagcagcatctttagtactcgctcgtaaacacactaattgttgtacttggtctgaacgcgtttataggttgtataatgaatgcgctcaggttttatctgaaactgtcttagctacaggtggtatatatgtgaaacctggtggtactagcagtggagatgctactactgcttatgcaaacagtgttttcaacataatacaagccacatctgctaatgttgcgcgtcttttgagtgttataacgcgtgatattgtatatgatgacattaagagcttgcagtatgaattgtaccagcaggtttataggcgagtcaattttgacccagcatttgttgaaaagttttattcttatttgtgtaagaatttctcattgatgatcttgtctgacgacggtgttgtttgttataacaacacattagccaaacaaggtcttgtagcagatatttctggttttagagaagttctctactatcagaacaatgtttttatggctgattctaaatgttgggttgaaccagatttagaaaaaggcccacatgaattttgttcacagcacacaatgttagtggaggttgatggtgagcctagatacttgccatatccagacccatcacgtattttgtgtgcatgtgtttttgtagatgatttggataagacagaatctgtggctgttatggagcgttatatcgctcttgccatagatgcgtacccactagtacatcatgaaaatgaggagtacaagaaggtattctttgtgcttctttcatacatcagaaaactctatcaagagctttctcagaatatgcttatggactactcttttgtaatggatatagacaagggtagtaaattttgggagcaggagttctatgaaaatatgtatagagcccctacaacattacagtcttgtggcgtttgtgtagtgtgtaatagtcaaactatattgcgctgtggtaattgtattcgcaaaccatttttgtgttgtaagtgttgctatgaccatgtcatgcacacagaccacaaaaatgttttgtctataaatccttacatttgctcacagccaggttgtggtgaagcagatgttactaaattgtacctcggaggtatgtcatacttctgcggtaatcataaaccaaagttatcaataccgttagtatctaatggtacagtgtttggaatttacagggctaattgtgcaggtagcgaaaatgttgatgattttaatcaactagctactactaattggtctactgtggaaccttatattttggcaaatcgttgtgtagattcgttgagacgctttgctgcagagacagtaaaagctacagaagaattacataagcaacaatttgctagtgcagaagtgagagaagtactctcagatcgtgaattgattctgtcttgggagccaggtaaaaccaggcctccattgaatagaaattatgttttcactggctttcactttactagaactagtaaagttcagctcggtgattttacatttgaaaaaggtgaaggtaaggacgttgtctattatcgagcgacgtctactgctaaattgtctgttggagacatttttgttttaacctcacacaatgttgtttctcttatagcgccaacgttgtgtcctcagcaaaccttttctaggtttgtgaatttaagacctaatgtgatggtacctgcgtgttttgtaaacaacattccattgtaccatttagtaggcaagcagaagcgtactacagtacaaggccctcctggcagtggtaaatcccattttgctataggattggcggcttactttagtaacgcccgtgtcgtttttactgcatgctctcatgcagctgttgatgctttatgtgaagaagcttttaagtttcttaaagtagatgattgcactcgtatagtacctcaaaggactactatcgattgcttctctaagtttaaagctaatgacacaggcaaaaagtacatttttagtactattaatgccttgccagaagttagttgtgacattcttttggttgacgaggttagtatgttgaccaattacgaattgtcttttattaatggtaagataaactatcaatatgttgtgtatgtaggtgatcctgctcaattaccggcgcctcgtacgttgcttaacggttcactctctccaaaggattataatgttgtcacaaaccttatggtttgtgttaaacctgacattttccttgcaaagtgttaccgttgtcctaaagaaattgtagatactgtttctactcttgtatatgatggaaagtttattgcaaataacccggaatcacgtcagtgtttcacggttatagttaataatggtaattctgatgtaggacatgaaagtggctcagcctacaacacaactcaattagaatttgtgaaagattttgtctgtcgcaataaggaatggcgggaagcaacattcatttcaccttataatgctatgaaccagagagcctaccgtatgcttggacttaatgttcagacagtagactcgtctcaaggttcggagtatgattatgttatcttttgtgttactgcagattcgcagcatgcactgaatattaacagattcaatgtagcgcttacaagagccaagcgtggtatactagttgtcatgcgtcagcgtgatgaactatattcagctcttaagtttatagagcttgatagtgtagcaagtctgcaaggtacaggcttgtttaaaatttgcaacaaagagtttagtggtgttcatccagcttatgcagtcacaactaaggctcttgctgcaacttataaagtaaatgatgaacttgctgcacttgttaacgtggaagctggttcagaaataacatataaacatcttatttctttgttagggtttaagatgagtgttaatgttgaaggctgccacaacatgtttataacacgtgatgaggctatccgcaacgtaagaggttgggtaggttttgatgtagaagcaacacatgcttgcggtactaacattggtactaacctgcctttccaagtaggtttctctactggtgcaggctttgtagttacgcctgagggacttgtagatacttcaataggcaataattttgagcctgtgaattctaaagcacctccaggtgaacaatttaatcacttgagagcgttattcaaaagtgctaaaccttggcatgttgtaaggccaaggattgtgcaaatgttagcggataacctatgcaacgtttcagattgtgtagtgtttgtcacgtggtgtcatggcctagaactaaccactttgcgctattttgttaaaataggcaaggaccaagtttgttcttgcggttctagagcaacaacttttaattcttatactcaggcttatgcttgttggaagcattgcttgggttttgattttgtttataatccactcttagtggatattcagcagtggggttattctggtaacctacaatttaaccatgatttgcattgtaatgtgcatggacacgcacatgtagcttctgcggatgctattatgacgcgttgtcttgcaattaataatgcattttgtcaagatgtcaactgggatttaacttaccctcatatagcaaatgaggatgaagtcaattctagctgtagatatttacaacgcatgtatcttaatgcatgtgttgatgctcttaaagttaacgttgtctatgatataggcaaccctaaaggtataaaatgtgttagacgtggagacttaaattttagattctatgataagaatccaatagtacccaatgtcaagcagtttgagtatgactataatcagcacaaagataagtttgctgatggtctttgtatgttttggaattgtaatgtggattgttatcccgacaattccttagtttgtaggtacgacacacgaaatttgagtgtgtttaacctacctggttgtaatggtggtagcttgtatgtcaacaagcatgcattccacacacctaaatttgatcgcactagctttcgtaatttgaaagctatgccattctttttctatgactcatcgccttgcgagaccattcaagtggatggagttgcgcaagaccttgtgtcattagctacgaaagattgtatcacaaaatgcaacataggcggtgctgtttgtaaaaagcacgcacaaatgtatgcagattttgtgacttcttataatgcagctgttactgctggttttactttttgggttactaataattttaacccatataatttgtggaaaagtttttcagctctccagtctatcgacaatattgcttataatatgtataagggtggtcattatgatgctattgcaggagaaatgcccactatcgtaactggagataaagtttttgttatagatcaaggcgtagaaaaagcagttttttttaatcaaacaattctgcctacatctgtagcgtttgagctgtatgcgaagagaaatattcgcacactgccaaacaaccgtattttgaaaggtttgggtgtagatgtgactaatggatttgtaatttgggattacacgaaccaaacaccactataccgtaatactgttaaggtatgtgcatatacagacatagaaccaaatggcctaatagtgctgtatgatgatagatatggtgattaccagtcttttctggctgctgataatgctgttttagtttctacacagtgttacaagcggtattcgtatgtagaaataccgtcaaacctgcttgttcagaacggtattccgttaaaagatggagcgaacctgtatgtttataagcgtgttaatggtgcgtttgttacgctacctaacacattaaacacacagggtcgcagttatgaaacttttgaacctcgtagtgatgttgagcgtgattttctcgacatgtctgaggagagttttgtagaaaagtatggtaaagaattaggtctacagcacatactgtatggtgaagttgataagccccaattaggtggtttacacactgttataggtatgtgcagacttttacgtgcgaataagttgaacgcaaagtctgttactaattctgattctgatgtcatgcaaaattattttgtattggcagacaatggttcctacaagcaagtgtgtactgttgtggatttgctgcttgatgatttcttagaacttcttaggaacatactgaaagagtatggtactaataagtctaaagttgtaacagtgtcaattgattaccatagcataaattttatgacttggtttgaagatggcattattaaaacatgttatccacagcttcaatcagcatggacgtgtggttataatatgcctgaactttacaaagttcagaattgtgttatggaaccttgcaacattcctaattatggtgttggaatagcgttgccaagtggtattatgatgaatgtggcaaagtatacacaactctgtcaatacctttcgaaaacaacaatgtgtgtaccgcatagtatgcgagtaatgcattttggagctggaagtgacaaaggagtggctccaggtagtactgttcttaaacaatggctcccagaagggacactccttgtcgataatgatattgtagactatgtgtctgatgcacatgtttctgtgctttcagattgcaataaatataagacagagcacaagtttgatcttgtgatatctgatatgtatacagacaatgattcaaaaagaaagcatgaaggcgtgatagccaataatggcaatgatgacgttttcatatatctctcaagttttctacgcaataatttggctctgggaggcagttttgctgtaaaattaacagagacaagttggcatgagagtttatatgacattgcacaggattgtgcatggtggacaatgttctgtacagcagtgaatgcatcttcttcagaagcattcctgattggtgttaattacttgggtgcaagtgcaaaggttaaagttagtggaaaaacactgcacgcaaattatatattttggaggaattgtaattatttacaaacctctgcttatagtatatttgatgttgctaagtttgatttgagattgaaagcaacgccagttgttaatttgaaaactgaacaaaagacagacttagtctttaatttaattaagtgtggtaagttactggtaagagatgttggtaacacctcttttactagtgactcttttgtgtgcactatgtagtgctgttttgtatgacagtagttcttacgtgtactactaccaaagtgccttcagaccacctgatggttggcatttacatgggggtgcgtatgcggttgttaatatttctagtgaatctaataatgcaggctcttcatctgggtgtactgttggtattattcatggtggtcgtgttgttaatgcttcttctatagctatgacggcaccgtcatcaggtatggcttggtctagcagtcagttttgtactgcatactgtaacttttcagatactacagtgtttgttacacattgttataaacatgttgggtgtcctataactggcatgcttcaacagcattctatacgtgtttctgctatgaaaaatggccagcctttttataatttaacagttagtgtagctaagtaccctacttttaaatcatttcagtgtgttaataatttaacatccgtatatttaaatggtgatcttgtttacacctctaatgagaccacagatgttacatctgcaggtgtttattttaaagctggtggacctataacttataaagttatgagagaagttagagccctggcttattttgttaatggtactgcacaagatgttattttgtgtgatgggccacctagaggcttgttagcatgccagtataatactggcaatttttcagatggcttttatccttttactaatagtagtttagttaagcagaagtttattgtctatcgtgaaaatagtgttaatactacttttacgttacgcaatttcacttttcataatgagactggcgccaaccctaatcctagtggtgttcagaatattcaaacttaccaaacacaaacagctcagagtggttattataattttaatttttcctttctgagtagttttgtttataaggagtctaattttatgtatggatcttatcacccaagttgtaattttagactagaaactattaataatggtttgtggtttaattcactttcagtttcaattgcttacggtcctcttcaaggtggttgcaagcaatctgtctttagtggtagagcaacctgttgttatgcttactcatatggaggtcctttgctgtgtaaaggtgtttattcaggtgagttaggtcataattttgaatgtggactgttagtttatgttactaagagcggtggctctcgtatacaaacagccactgaaccgccagttataactcaacacaattataataatattactttaaatacttgtgttgattataatatatatggcagaactggccaaggttttattactaatgtaaccgactcagctgttagttataattatctagcagacgcaggtttggctattttagatacatctggttccatagacatctttgttgtacaaggtgaatatggtcttacttattataaggttaacccttgcgaagatgtcaaccagcagtttgtagtttctggtggtaaattagtaggtattcttacttcacgtaatgagactggttctcagcttcttgagaaccagttttacattaaaatcactaatggaacacgtcgttttagacgttctattactgaaaatgttgaaaattgcccttatgttagttatggtaagttttgtataaaacctgatggttcaattgccacaatagtaccaaaacaattggaacagtttgtggcacctttacttaatgttactgaaaatgtgctcatacctaacagttttaatttaactgttacagatgagtacatacaaacgcgtatggataaggtccaaattaattgtctgcagtatgtttgtggcaattctctggattgtagagatttgtttcgacaatatgggcatgtttgtgacaacatattgtctgtagtaaatagtattggtcaaaaagaagatatggaacttttgaatttctattcttctactaaaccggttggttttaatacaccatttcttagtaatgttagcactggtgagtttaatatttctcttctgttaacaactcctagtagtcctagaaggcgttcttttattgaagaccttctatttacaagcgttgaatctgttggattaccaacagatgacgcatacaaaaattgcactgcaggacctttaggttttcttaaggaccttgcgtgtgctcgtgaatataatggtttgcttgtgttgcctcccattataacagcagaaatgcaaactttgtatactagttctctagtagcttctatggcttttggtggtattactgcagctggtgctataccttttgccacacaactgcaggctagaattaatcacttgggtattacccagtcacttttgttgaagaatcaagaaaaaattgctgcttcctttaataaggccattggtcgtatgcaggaaggttttagaagtacatctttagcattacaacaaattcaagatgttgttaataagcagagtgctattcttactgagactatggcatcacttaataaaaattttggtgccatttcttctgtgattcaagaaatctaccagcaacttgacgccatacaagcaaatgctcaagtggatcgtcttataactggtagattgtcatcactttctgttttagcatctgctaagcaggcggagtatattagagtgtcacaacagcgtgagttagctactcagaagattaatgagtgtgttaagtcacagtccattaggtactccttttgtggtaatggacgacatgttttaaccataccgcaaaatgcacctaatggtatagtgtttatacacttttcttacactccagatagttttgttaatgttactgcaatagtgggtttttgtgtaaagccagctaatgctagtcagtatgcaatagtacccgctaatggtaggggtattttcatacaagttaatggtagttactacatcactgcacgagatatgtatatgccaagagctattactgcaggagatatagttacgcttacttcttgtcaagtaaattatgtaagtgtaaataagaccgtcattactacattcgtagacaatgatgattttgattttaatgacgaattgtcaaaatggtggaatgatactaagcatgagctaccagactttgacaaattcaattacacagtacctatacttgacattgatagtgaaattgatcgtattcaaggcgttatacagggtcttaatgactctctaatagaccttgaaaaactttcaatactcaaaacttatattaagtggccttggtatgtgtggttagccatagcttttgccactattatcttcatcttaatattaggatgggttttcttcatgactgggtgttgtggttgttgttgtggatgctttggcattatgcctctaatgagtaagtgtggtaagaaatcttcttattacacgacttttgataacgatgtggtaactgaacaatacagacctaaaaagtctgtttaatgatccaaagtcccacgtccttcttaatagtattaattttgctttggtgtaaacttgtactaagttgttttagagagtttattatagcgcttcaacaactaacacaagttttactccaaattatcgatagtaatttacagtctagactgaccctttgtcacagtctagactaatgttaaacttagaagcaattattgaaactggtgagcaagtgattcaaaaaatcagtttcaatttacagcatatttcaagtgtattaaacacagaagtatttgacccctttgactattgttattacagaggaggtaatttttgggaaatagagtcagctgaagattgttcaggtgatgatgaatttattgaataagtcgctagaggaaaatggaaattttctaacagcgctttacatatttgtaggatttttagcactttatcttctaggtagagcacttcaagcatttgtacaggctgctgatgcttgttgtttattttggtatacatgggtagtaattccaggagctaagggtacagcctttgtatacaagtatacatatggtagaaaacttaacaatccggaattagaagcagttattgtcaacgagtttcctaagaacggttggaataataaaaatccagcaaattttcaagatgtccaacgagacaaattgtactcttgactttgaacagtcagttgagctttttaaagagtataatttatttataactgcattcttgttgttcttaaccataatacttcagtatggctatgcaacaagaggtaagtttatttatatactgaaaatgatagtgttatggtgcttttggccccttaacattgcagtaggtgtaatttcatgtatatacccaccaaacacaggaggtcttgtcgcagcgataatacttacagtgtttgcgtgtctgtcttttgtaggttattggatccagagtattagactctttaagcggtgtaggtcatggtggtcatttaacccagaatctaatgccgtaggttcaatactcctaactaatggtcaacaatgtaattttgctatagagagtgtgccaatggtgctttctccaattataaagaatggtgttctttattgtgagggtcagtggcttgctaagtgtgaaccagaccacttgcctaaagatatatttgtttgtacaccggatagacgtaatatctaccgtatggtgcagaaatatactggtgaccaaagcggaaataagaaaaggtttgctacgtttgtctatgcaaagcagtcagtagacactggcgagctagaaagtgtagcaacaggtggaagtagcctttacacataaatgtgtgtgtgtagagagtatttaaaattattcttcaatagtgcctctattttaagagcgcggaagagtatttgttttgaggatattaatataaatcctctttgttttgtactctctttacaagagttattatttaagcaacagtttttcctttcctttgtttggaagaaagttgttgttaatggtgtagaattccaagtagaaaatggaaaagtccactacgaaggaaaccccattttctaaaaaggttgttgtaggttgtggtcccattataagaaggattaaatggattaaaccacctacactacttacttgtaataagggcgtttggacttacaagcgcttaacaaatacagacgatgaaatggctgactagttttggaagagcagttatttcttgttataaagccctactattaactcagttaagagtattagataggttaattttagatcacggaccaaaacgcgtcttaacgtgtggtaggcgagtgcttttatctcaattagatctagtttataggttggcatatacgcccacccaatcgctggtatgaataatagtaaagataatccttttcgcggagcaatagcaagaaaagcgcgaatttatctgagagaaggattagagtgtgtttactttcttaacaaagcaggacaagcagagtcttgtcccgcgtgtacctctctagtattccaggggaaaacttgtgaggaacacaaagataataataatcttttgtcatggcaagcggtaaggcaactggaaagacagatgccccaactccagtcatcaaactaggaggaccaaagccacctaaagttggttcttctggaaatgtatcttggtttcaagcaataaaagccaagaagttaaattcacctccgcctaagtttgaaggtagcggtgttcctgataatgaaaatctaaaaccaagtcagcagcatggatattggagacgccaagctaggtttaagccaggtaaaggtggaagaaaaccagtcccagatgcttggtatttttactatactggaacaggaccagccgctaacctgaattggggtgatagccaagatggtatagtgtgggttgctggtaagggtgctgatactaaatttagatctaatcagggtactcgtgactctgacaagtttgaccaatatccgctacggttttcagacggaggacctgatggtaatttccgttgggatttcattcctctgaatcgtggtaggagtggaagatcaacagcggcttcatcagcagcatctagtagagcaccgtcgcgtgatggctcgcgtggacgtagaagcggagctgaagatgatcttatagctcgtgcagcaaagatcattcaggatcagcagaagaagggttctcgcattactaaagctaaggccgatgaaatggctcatcgccggtattgtaagcgtactatcccacctggttataaggttgatcaagtatttggtccccgtactaaatgtaaggagggaaattttggtgatgacaagatgaatgaggagggtattaaggatgggcgcgttacagcaatgctcaacctagtccctagcagccatgcttgtctttttggaagtagagtgacgcccaaacttcaaccagatgggctgcacttgagatttgaatttactactgtggtttcacgtgatgatccgcagtttgataattatgtgaaaatttgtgatcagtgtgtcgatggtgtagggacgcgtccaaaagacgatgaaccgagaccaaagtcacgcccaaattcaagacctgctacaagaacaagttctccagcgccaagacaacagcgtcaaaagaaggagaagaagtcaaagaagcaggatgatgaagtagataaggcattgacctcagatgaggagaggaacaatgcacagctggaatttgatgatgaacccaaggtgattaactggggggagtcagcacttggagagaatgagttgtaaagctagatttccaacttaacatcatggacgtgcgtatgctgtttttccctactatagactttttagcatattattttttgctatttgtatggtttattacaggtgaagattgtatgtatttgttgtacactcgtatgttctatattatgttttctgtagttgttattagtgttgttcttgttcttactctactgttctcttttctttattttagagtatcaataagaatcaaggaagataggcatgtagtttgattacctacatgtctatcgccagggaaatgtctaatctgtctacttagtagcctggaaacgaacggtagacccttagattttaatttagtttaatttttagtttagtttaagttagtttagagtaggtataaagaagccagtgccggggccacgcggagtacgatcgagggtacagcactaggacgcccattaggggaagagctaaattttagtttaagttaagtttaataggctatgtatagttaaaatttataggctagtatagagttagagcaaaaaaaaaaaaaaaa

SEQ ID NO: 4 - protein, 1162 aa, Infectious bronchitis virus
MLVTPLLLVTLLCALCSAVLYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHVGCSLTGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGGIGQGFITNVTDSAVSYNYLADAGLAILDTSGSIDIFVVQSEYGLNYYKVNPCEDVNQQFVVSGGKLVGILTSRNETGSQLLENQFYIKITNGTRRFRRSITESVENCPYVSYGKFCIKPDGSIATIVPKQLEQFVAPLLNVTENVLIPNSFNLTVTDEYIQTLMDKVQINCLQYICGNSLECRNLFQQYGPVCDNILSVVNSVGQKEDMELLNFYSSTKPAGFNTPVLSNVSTGEFNISLFLTTPSSPRRRSFIEDLLFTSVESVGLPTDDAYKNCTAGPLGFLKDLVCAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQVNYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 5 - protein, 1162 aa, Infectious bronchitis virus
MLVTPLLLVTLLCALCSAALYDSSSYVYYYQSAFRPPDGWHLHGGAYAVVNISSESNNAGSSSGCTVGIIHGGRVVNASSIAMTAPSSGMAWSSSQFCTAYCNFSDTTVFVTHCYKHGGCPITGMLQQHSIRVSAMKNGQLFYNLTVSVAKYPTFKSFQCVNNLTSVYLNGDLVYTSNETTDVTSAGVYFKAGGPITYKVMREVRALAYFVNGTAQDVILCDGSPRGLLACQYNTGNFSDGFYPFTNSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPLLCKGVYSGELDHNFECGLLVYVTKSGGSRIQTATEPPVITQHNYNNITLNTCVDYNIYGRTGQGFITNVTDSAVSYNYLADAGLAILDTSGSIDIFVVQSEYGLNYYKVNPCEDVNQQFVVSGGKLVGILTSRNETGSQLLENQFYIKITNGTRRFRRSITESVENCPYVSYGKFCIKPDGSIATIVPKQLEQFVAPLLNVTENVLIPNSFNLTVTDEYIQTRMDKVQINCLQYICGNSLECRNLFQQYGPVCDNILSVVNSVGQKEDMELLNFYSSTKPAGFNTPVLSNVSTGEFNITLFLTTPSSPRRRSFIEDLLFTSVESVGLPTDDAYKNCTAGPLGFLKDLACAREYNGLLVLPPIITAEMQTLYTSSLVASMAFGGITAAGAIPFATQLQARINHLGITQSLLLKNQEKIAASFNKAIGHMQEGFRSTSLALQQIQDVVNKQSAILTETMASLNKNFGAISSVIQEIYQQLDAIQANAQVDRLITGRLSSLSVLASAKQAEYIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQYRPKKSV

SEQ ID NO: 6 - protein, 409 aa, Infectious bronchitis virus
MASGKATGKTDAPTPVIKLGGPKPPKVGSSGNVSWFQAIKAKKLNSPPPKFEGSGVPDNENLKPSQQHGYWRRQARFKPGKGGRKPVPDAWYFYYTGTGPAANLNWGDSQDGIVWVAGKGADTKFRSNQGTRDSDKFDQYPLRFSDGGPDGNFRWDFIPLNRGRSGRSTAASSAASSRAPSREVSRGRRSGSEDDLIARAARIIQDQQKKGSRITKAKADEMAHRRYCKRTIPPNYKVDQVFGPRTKGKEGNLGDDKMNEEGIKDGRVTAMLNLVPSSHACLFGSRVTPRLQPDGLHLKFEFTTVVPRDDPQFDNYVKICDQCVDGVGTRPKDDEPRPKSRSSSRPATRGNSPAPRQQRPKKEKKPKKQDDEVDKALTSDEERNNAQLEFDDEPKVINWGDSALGENEL

SEQ ID NO: 7 - protein, 409 aa, Infectious bronchitis virus
MASGKAAGKTDAPTPVIKLGGPKPPKVGSSGNVSWFQAIKAKKLNSPPPKFEGSGVPDNENLKPSQQHGYWRRQARFKPGKGGRKPVPDAWYFYYTGTGPAANLNWGDSQDGIVWVAGKGADTKFRSNQGTRDSDKFDQYPLRFSDGGPDGNFRWDFIPLNRGRSGRSTAASSAASSRAPSREVSRGRRSGSEDDLIARAARIIQDQQKKGSRITKAKADEMAHRRYCKRTIPPNYKVDQVFGPRTKGKEGNFGDDKMNEEGIKDGRVTAMLNLVPSSHACLFGSRVTPRLQPDGLHLKFEFTTVVPRDDPQFDNYVKICDQCVDGVGTRPKDDEPRPKSRSSSRPATRGNSPAPRQQRPKKEKKPKKQDDEVDKALTSDEERNNAQLEFDDEPKVINWGDSALGENEL

SEQ ID NO: 8 - protein, 409 aa, Infectious bronchitis virus
MASGKTTGKTDAPAPVIKLGGPKPPKVGSSGNASWFQALKAKKLNSPPPKFEGSGVPDNENLKLSQQHGYWRRQARYKPGKGGRKSVPDAWYFYYTGTGPAADLNWGDSQDGIVWVSAKGADTKSRSNQGTRDPDKFDQYPLRFSDGGPDGNFRWDFIPINRGRSGRSTAASSAASSRAPSRDGSRGRRSGAEDDLIARAAKIIQDQQKKGSRITKAKADEMAHRRYCKRTIPPGYKVDQVFGPRTKGKEGNFGDDKMNEEGIKDGRVTAMLNLVPSSHACLFGSRVTPKLQPDGLHLRFEFTTVVSRDDPQFDNYVKICDQCVDGVGTRPKDDEPRPKSRPNSRPATRTSSPAPRQQRQKKEKKSKKQDDEVDKALTSDEERNNAQLEFDDEPKVINWGDSALGENEL

SEQ ID NO: 9 - protein, 109 aa, Infectious bronchitis virus
MMNLLNKSLEENGSFLTALYIFVGFLAFYLLGRALQAFVQAADACCLFWYTWLVIPGVKGTAFVYKYTYGRKLNNSELEAVVVNEFPKNGWNNKNPANFQDVQRNKLYS

SEQ ID NO: 10 - protein, 110 aa, Infectious bronchitis virus
VMMNLLNKSLEENGSFLTALYIFVGFLAFYLLGRALQAFVQAADACCLFWYTWLVIPGVKGTAFVYKYTYGRKLNNSELEAVVVNEFPKNGWNNKNPANFQDVQRNKLYS

SEQ ID NO: 11 - protein, 225 aa, Infectious bronchitis virus
MSNETNCTLDFEQSVELFKEYNLFITAFLLFLTIILQYGYATRSKFIYILKMIVLWCFWPLNIAVGVISCIYPPNTGGLVAAIILTVFACLSFVGYWIQSIRLFKRCRSWWSFNPESNAVGSILLTNGQQCNFAIESVPMVLSPIIKNGVLYCEGQWLAKCEPDHLPKDIFVCTPDRRNIYRMVQKYTGDQSGNKKRFATFVYAKQSVDTGELESVATGGSSLYT

SEQ ID NO: 12 - protein, 225 aa, Infectious bronchitis virus; identical to SEQ ID NO: 11

SEQ ID NO: 13 - DNA, 11944 bp, synthetic construct
  source 1..11944: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatcggccgcccgggtaatacgactcactatagggacttaagatagatattaatatatatctattgcactagccttgcgctagatttcaaacttaacaaaacggacttaaatacctacagctggtccccataggtgttccattgcagtgcactttagtgccctggatggcacctggccacctgtcaggtttttgttgttaaaatatcattgttgctggtatcactgcttgttttgccgtgtctcactttatacatccgttgcttgggctacctagtatccagcgtcctacgggcgccgtggtcggttcgagtgcgaaggacctctggttcatctagcggtaggcgggtgtgtggaagtagcgcttcagacgtactggttctgttgcgtgaaacgcggggtcacctccccccacatacctctaagggcttttgagcctagcgttgggctacgttctcgcacaaggtcggctatacgacgtttgtagggggtagtgccaaacaacccctgaggtgacaggttctggtggtgtttcgaaaacaacaatgtgtgtgccgcataatatgcgcgttatgcattttggagcaggtagtgataaaggagtggcgcctggtagtactgttcttaaacagtggctccccgaaggaacactccttgtcgataatgatattgtagattatgtttctgacgcacacgtttctgtgctttcagattgcaataaatataagacagagcacaagtttgatcttgtgatatctgatatgtatacagacaatgattcaaaaagaaagcatgaaggcgtgatagccaataatggcaatgatgacgtcttcatatacctttcaagttttctacgcaataatttggctctgggaggcagttttgctgtaaaattaacagagacaagttggcatgagagtttatatgacattgcacaggattgtgcatggtggacaatgttttgtacagcagtgaatgcatcttcttcagaagcattcctgattggtgttaattacttgggtgcaagtgcaaaggttaaagttagtggaaaaacactgcacgcaaattatatattttggaggaattgtaattatttacaaacctctgcttatagtatatttgatgttgctaagtttgatttgagattgaaagcaacgccagttgttaatcttaagactgaacaaaagacagacttagtctttaatttgataaagtgtggtaagttactggtaagagatgttggtaacacctcttttactagtgactcttttgtgtgcactatgtagtgctgctttgtatgactcgagttatattggtgattttagatgtatccagcttgtgaactcaaacggtgctaatgttagtgctccaagcattagcactgagaccgttgaagtttcacaaggcctggggacatattatgtgttagatcgagtttatttaaatgccacattattgcttactggttactacccggtcgatggttctaagtttagaaacctcgctcttacgggaactaactcagttagcttgtcgtggtttcaaccaccctatttaagtcagtttaatgatggcatatttgcgaaggtgcagaaccttaagacaagtacgccatcaggtgcaactgcatattttcctactatagttataggtagtttgtttggctatacttcctataccgttgtaatagagccatataatggtgttataatggcctcagtgtgccagtataccatttgtcagttaccttacactgattgtaagcctaacactaatggtaataagcttatagggttttggcacacggatgtaaaacccccaatttgtgtgttaaagcgaaatttcacgcttaatgttaatgctgatgcattttattttcatttttaccaacatggtggtactttttatgcgtactatgcggataaaccctccgctactacgtttttgtttagtgtatatattggcgatattttaacacagtattatgtgttacctttcatctgcaacccaacagctggtagcacttttgctccgcgctattgggttacacctttggttaagcgccaatatttgtttaatttcaaccagaagggtgtcattactagtgctgttgattgtgctagtagttataccagtgaaataaaatgtaagacccagagcatgttacctagcactggtgtctatgagttatccggttatacggtccaaccagttggagttgtataccggcgtgttgctaacctcccagcttgtaatatagaggagtggcttactgctaggtcagtcccctcccctctcaactgggagcgtaagacttttcagaattgtaattttaatttaagcagcctgttacgttatgttcaggctgagagtttgttttgtaataatatcgatgcttccaaagtgtatggcaggtgctttggtagtatttcagttgataagtttgctgtaccccgaagtaggcaagttgatttacagcttggtaactctggatttctgcagactgctaattataagattgatacagctgccacttcgtgtcagctgcattacaccttgcctaagaataatgtcaccataaacaaccataacccctcgtcttggaataggaggtatggctttaatgatgctggcgtctttggcaaaaaccaacatgacgttgtttacgctcagcaatgttttactgtaagatctagttattgcccgtgtgctcaaccggacatagttagcccttgcactactcagactaagcctaagtctgcttttgttaatgtgggtgaccattgtgaaggcttaggtgttttagaagataattgtggcaatgctgatccacataagggttgtatctgtgccaacaattcatttattggatggtcacatgatacctgccttgttaatgatcgctgccaaatttttgctaatatattgttaaatggcattaatagtggtaccacatgttccacagatttgcagttgcctaatactgaagtggttactggcatttgtgtcaaatatgacctctacggtattactggacaaggtgtttttaaagaggttaaggctgactattataatagctggcaaacccttctgtatgatgttaatggtaatttgaatggttttcgtgatcttaccactaacaagacttatacgataaggagctgttatagtggccgtgtttctgctgcatttcataaagatgcacccgaaccggctctgctctatcgtaatataaattgtagctatgtttttagcaataatatttcccgtgaggagaacccacttaattactttgatagttatttgggttgtgttgttaatgctgataaccgcacggatgaggcgcttcctaattgtgatctccgtatgggtgctggcttatgcgttgattattcaaaatcacgcagggctcaccgatcagtttctactggctatcggttaactacatttgagccatacactccgatgttagttaatgatagtgtccaatccgttgatggattatatgagatgcaaataccaaccaattttactattgggcaccatgaggagttcattcaaactagatctccaaaggtgactatagattgtgctgcatttgtctgtggtgataacactgcatgcaggcagcagttggttgagtatggctctttctgtgttaatgttaatgccattcttaatgaggttaataacctcttggataatatgcaactacaagttgctagtgcattaatgcagggtgttactataagttcgagactgccagacggcatctcaggccctatagatgacattaattttagtcctctacttggatgcataggttcaacatgtgctgaagacggcaatggacctagtgcaatccgagggcgttctgctatagaggatttgttatttgacaaggtcaaattatctgatgttggctttgtcgaggcttataataattgcaccggtggtcaagaagttcgtgacctcctttgtgtacaatcttttaatggcatcaaagtattacctcctgtgttgtcagagagtcagatctctggctacacaaccggtgctactgcggcagctatgttcccaccgtggtcagcagctgccggtgtgccatttagtttaagtgttcaatatagaattaatggtttaggtgtcactatgaatgtgcttagtgagaaccaaaagatgattgctagtgcttttaacaatgcgctgggtgctatccaggatgggtttgatgcaaccaattctgctttaggtaagatccagtccgttgttaatgcaaatgctgaagcactcaataacttactaaatcaactttctaacaggtttggtgctattagtgcttctttacaagaaattctaactcggcttgaggctgtagaagcaaaagcccagatagatcgtcttattaatggcaggttaactgcacttaatgcgtatatatccaagcaacttagtgatagtacgcttattaaagttagtgctgctcaggccatagaaaaggtcaatgagtgcgttaagagccaaaccacgcgtattaatttctgtggcaatggtaatcatatattatctcttgtccagaatgcgccttatggcttatattttatacacttcagctatgtgccaatatcctttacaaccgcaaatgtgagtcctggactttgcatttctggtgatagaggattagcacctaaagctggatattttgttcaagatgatggagaatggaagttcacaggcagttcatattactaccctgaacccattacagataaaaacagtgtcattatgagtagttgcgcagtaaactacacaaaggcacctgaagttttcttgaacacttcaatacctaatccacccgactttaaggaggagttagataaatggtttaagaatcagacgtctattgcgcctgatttatctctcgatttcgagaagttaaatgttactttgctggacctgacgtatgagatgaacaggattcaggatgcaattaagaagttaaatgagagctacatcaacctcaaggaagttggcacatatgaaatgtatgtgaaatggccttggtatgtgtggctagccatagcttttgccactattatcttcatcttaatattaggatgggttttcttcatgactgggtgttgtggttgttgttgtggatgctttggcattatgcctctaatgagtaagtgtggtaagaaatcttcttattacacgacttttgataacgatgtggtaactgaacaatacagacctaaaaagtctgtttaatgatccaaagtcccactagtttcttaatagtattaattttgctttggtgtaaacttgtactaagttgttttagagagtttattattgcccttcaacaactaacacaagttttactccaaattatcgatagtaatttacagtctagactgaccctttggcacagtctagactaatgttaaacttagaagcaattattgaaaccggtgatcaagtgattcaaaaaatcagtttcaatttacagcatatttcaagtgtattaaacacagaagtatttgacccctttgactattgttattacagaggaggtaatttttgggaaatagagtcagctgaagattgttcaggtgatgatgaatttattgaataagtcgctagaggagaatggaagttttctaacggcactttacatatttgtaggatttttagcattttatcttctaggtagagcacttcaagcatttgtacaggctgctgatgcttgttgtttattttggtacacgtggttagtaattccaggagttaagggtacagcctttgtatacaagtatacatatggtagaaaacttaacaattcggaattagaagcagttgttgttaacgagtttcctaagaacggttggaataataaaaatccagcaaattttcaagatgtccaacgaaacaaattgtactcttgactttgaacagtcagttgagctttttaaagagtataatttatttataactgcattcttgttgttcttaaccataatacttcagtatggttatgcaacgcgtagtaagtttatttatatacttaaaatgatagtgttatggtgcttttggccccttaacattgcagtaggtgtaatttcatgtatatacccaccaaacacaggaggtcttgtcgcagcgataatacttactgtgtttgcgtgtctttcttttgtaggttattggatccagagtattagactctttaagcggtgtagatcttggtggtcatttaacccagaatctaacgccgtaggttcaatactcctaactaatggtcaacaatgtaattttgctatagagagtgtgccgatggtgctttctcctattataaagaatggtgttctttattgtgagggtcagtggcttgctaaatgtgaaccagaccacttgcctaaagacatatttgtatgcacaccagatagacgtaatatctatcgtatggtgcagaaatacactggtgaccaaagcggaaataagaaaaggtttgctacatttgtctatgcaaagcagtcagtagacactggcgagctagaaagtgtagcaacaggtggaagtagcctttacacataaatgtgtgtgtgtagagagtatttaaaattattcttcaatagtgcctctattttaagagcgcggaagagtatttgttttgaggatattaatataaatcctctttgttttgtactctctttacaagagttattatttaagcaacagtttttcctttcctttgtttggaagaaagttgttgttaatggtgtagaattccaagtagaaaatggaaaagtccactacgaaggaaaccccattttccaaaaaggttgttgtaggttgtggtcccattataagaaggattaaatggattaaaccacctacactacttacttgtaataagggcgtttggacttacaagcgcttaacaaatacagacgatgaaatggctgactagttttggaagagcagttatttcttgttataaagccctactattaactcagttaagagtattagataggttaattttagatcacggaccaaagcgagtcttaacgtgtggtaggcgagtgcttttatctcaattagatttagtttataggttggcatatacgcccacccaatcgctggtatgaataatagtaaagataatccttttcgcggagcaatagcaagaaaagcgcgaatttatctgagagaaggattagagtgtgtttactttcttaacaaagcaggacaagcagagccttgtcccgcgtgtacctccctagtatttcaggggaaaacttgtgaggaacacacagataataataatcttttgtcatggcgagcggtaagacaactgggaagacagacgccccagcgccagtcatcaaactaggagggccaaaaccacctaaagttggttcttctggaaatgctagctggtttcaagcactaaaagccaagaagttaaattcacctcctcctaagtttgaaggtagcggcgttcctgataatgaaaatcttaaattaagccagcaacatgggtactggagacgtcaagccaggtacaagccaggtaaaggcggaagaaaatcagtcccagatgcttggtacttctattacactggaacaggaccagccgctgacctgaattggggtgatagccaagatggtatagtgtgggtttctgcaaagggtgctgatactaaatctagatctaaccagggtacaagggatcctgataagtttgaccaatacccgctacgattctcagatggaggacctgatggtaatttccgttgggacttcattccaataaatcgtggtaggagtggaagatcaacagcggcttcatcagcagcatctagtagagcaccgtcgcgtgatggctcgcgtggacgtagaagcggagctgaagatgatcttatagctcgtgcagcaaagatcattcaggatcagcagaagaagggttctcgcattactaaagctaaggccgatgaaatggctcatcgccggtattgtaagcgtactatcccacctggttataaggttgatcaagtatttggtccccgtactaaaggtaaggagggaaattttggtgatgacaagatgaatgaggagggtattaaggatgggcgcgttacagcaatgctcaacctagtccctagcagccatgcttgtctttttggaagtagagtgacgcccaaacttcaaccagatgggctgcacttgagatttgaatttactactgtggtttctagggatgatccgcagtttgataattatgtgaaaatttgtgatcagtgtgtcgatggtgtagggactcggccaaaagacgatgaaccgagaccaaagtcacgcccaaattcaagacctgctacaagaacaagttctccagcgccaagacaacagcgtcaaaagaaggagaagaagtcaaagaagcaggatgatgaagtagataaggcattgacctcagatgaggagaggaacaatgcacagctggaatttgatgatgaaccgaaagtgattaactggggggattcagcacttggagagaatgagttgtaaagctagatttccaacttaacatcatggacgtgcgtatgctgtttttccctactatagactttttagcatattattttttgctatttgtatggtttattacaggtgaagattgtatgtatttgttgtacactcgtatgttctatattatgttttctgtagttgttattagtgttgttcttgttcttactctactgttctcttttctttattttagagtatcaataagaatcaaggaagataggcatgtagtttgattacctacatgtctatcgccagggaaatgtctaatctgtctacttagtagcctggaaacgaacggtagacccttagattttaatttagtttaatttttagtttagtttaagttagtttagagtaggtataaagaagccagtgccggggccacgcggagtacgatcgagggtacagcactaggacgcccactaggggaagagctaaattttagtttaagttaagtttaattggctaagtatagttaaaatttataggctagtatagagttagagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagtttaaacttaattaagaattcccttggctcgagttcgaaatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 14086 bp, synthetic construct
  source 1..14086: mol_type = other DNA
gcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagattaatacgactcactatagggacttaagatagatattaatatatatctattgcactagccttgcgctagatttcaaacttaacaaaacggacttaaatacctacagctggtccccataggtgttccattgcagtgcactttagtgccctggatggcacctggccacctgtcaggtttttgttgttaaaatatcattgttgctggtatcactgcttgttttgccgtgtctcactttatacatccgttgcttgggctacctagtatccagcgtcctacgggcgccgtggtcggttcgagtgcgaaggacctctggttcatctagcggtaggcgggtgtgtggaagtagcgcttcagacgtactggttctgttgcgtgaaacgcggggtcacctccccccacatacctctaagggcttttgagcctagcgttgggctacgttctcgcacaaggtcggctatacgacgtttgtagggggtagtgccaaacaacccctgaggtgacaggttctggtggtgtttcgaaaacaacaatgtgtgtgccgcataatatgcgcgttatgcattttggagcaggtagtgataaaggagtggcgcctggtagtactgttcttaaacagtggctccccgaaggaacactccttgtcgataatgatattgtagattatgtttctgacgcacacgtttctgtgctttcagattgcaataaatataagacagagcacaagtttgatcttgtgatatctgatatgtatacagacaatgattcaaaaagaaagcatgaaggcgtgatagccaataatggcaatgatgacgtcttcatatacctttcaagttttctacgcaataatttggctctgggaggcagttttgctgtaaaattaacagagacaagttggcatgagagtttatatgacattgcacaggattgtgcatggtggacaatgttttgtacagcagtgaatgcatcttcttcagaagcattcctgattggtgttaattacttgggtgcaagtgcaaaggttaaagttagtggaaaaacactgcacgcaaattatatattttggaggaattgtaattatttacaaacctctgcttatagtatatttgatgttgctaagtttgatttgagattgaaagcaacgccagttgttaatcttaagactgaacaaaagacagacttagtctttaatttgataaagtgtggtaagttactggtaagagatgtcggtaacacgtcttttattagtgactcttttgtttgcactatgtagtgctgttttgtatgatagtaattcttatgtgtattattatcagagtgcttttaggccaggaagtggttggcatttacatggaggtgcttatgcagtagttaatgtgtctgagcaaaccagccctggcagttgcactgctggtgctattggatacagtatgaatttcagtgcagcctcagtagccatgactgcaccattaaatggtatgtcatggtcagatagacaattctgtatggctcactgcaattttactaatattgtagtgtttgttacacactgctttaaaaggggacagcagttttgtcctttaacaggttttatagagcaaaactatattcgtattgctgctatgagaattaatggtactggtcctagagacttattttataattcaacagtttctgtgagtaagtaccctaaatttaggtcactccaatgtgttaataatcaaacatctgtatatttaaatggtgatcttgtttttacttctaatgagactttatatgtctcaggtgctggtgttgattttacagctggtggacccataacttataaagttatgcaggaagtcaaagccctggcttattttgttaatggtaccgcacacgatgttattttgtgcgatggtacacccagaggtttgttagcatgccaatataatactggcaattttacagatggcttctatccttttactaatagtagtttagttaaggaaaggtttattgtctatcgtgagaatagtgttaataccacttgtgttttacataattttacgttttttaatgagactagtgcaccgcctgccggcggtgatattagtgctagatttccaacttatcaaacagtaacagctcagagtggttattataattttaatttttcatttctgagtggttttgtttataaggcaagcgattttatgtatgggtcttaccacccaaagtgtaattttagaccagaaactcttaataatgacctctggtttaattcactgtcaatctcacttgcttatggcccactacaagggggctgtaagcagtcggtttttaataatagagcaacgtgttgttatgcttattcatacaacggtcctcgtgcttgtaaaggtgtctatcgaggtcagttacaacaattgtttgaatgtgggttgctagtttatattactaagagcgatggctctcgtatacaaacaacaacaaaaccacatgtattaactacacatttctataataacatcactttagataagtgtgttgattataatatatatggtagagtaggccaaggttttataactaatgtaaccgactcaactgctgcttataattatttagcagatggagggttagctattttagacacatcaggtgctatagacattttcgttgtacaaggtgaatatggccttaatttttacaaggttaatccttgcgaagatgttaatcagcagtttgtagtttctggtggtaaattagtaggtattcttacttcacgtaatgtcactgattctcagtttcttgagaaccagttttacattaaactcactaatgaaacacatcgttctagacgttctgttagtgaaaatgttacaagttgtccgtatgttagttatggcaagttttgtataaaacctgatggttcaatttctgtaatagtaccaaaagaactggaacagtttgtggcacctttactcaatgtaactgaatatgtgctcatacctgatagttttaatttgactgtcacagatgagtacatacaaacacgtatggacaaggttcaaataaattgccttcaatatgtttgtggcaattctttggagtgtagaaagttatttcaacaatatggtcctgtctgtgacaacatattgtccgtcgttaatagtgttggtcaaaaggaagatatggagcttttaagtttctattcttctactaagccaacgggttataatacaccaatttttaataatttaacaactggtgcatttaatgtttcacttcttctaccgcaaccaggtagtccaagaggccgatctttcgtagaagatttgttatttactagtgttgagactgttggattaccgactgacgctgagtataagaagtgcacagcaggacctttgggtactctcaaagatcttatctgtgctcgtgaatataatggcctattagtgttgcctccaattattacagcggatatgcaaacaatgtataccgcttctttagtaggtgctatggcttttggtggtattacatcagctgcagctataccttttgccactcagattcaggcacgaattaatcatcttggtattacacagtctttgttaatgaaaaaccaagaaaagattgctgcttcctttaataaggccattggccaaatgcaggaaggttttaggagtacatctctagcattacaacaaattcaagatgttgttaataagcagagtgctattcttactgagaccatggcatcacttaataaaaattttggtgctatttcttctgtgattcaagacatttaccagcaacttgatgtcatacaagcagacgctcaagtggatcgacttataactggtagattgtcgtcactttctgtcttagcatctgctaagcagtctgaatacattatagcatcacaacagcgtgaattagctactcagaaaattaatgagtgtgttaaatcacagtctactaggtattccttttgtggtaatggacgacatgttctaaccataccacagaatgcacctaatggtatagtgtttatacactttacttatacaccagagagttttgttaatgttacagcaatagtagggttttgtgtaaacccagctaatgctagtcagtatgcaatagtgcccgctaatggtaggggtatttttatacaagttaatggtacttactatatcacagcacgtgatatgtatatgccaagaagcattacagcaggagacatagttactttgacttcttgtcaagcaaactatgttagtgttaataaaacagtcattactacctttgtagaagatgacgattttaattttgatgacgagttattaaaatggtggaatgaaactaagcatgatttgccagactttgatcagtttaattacaccattcccgttttaaatataacttatgacattgacaagattgaggaagttattaagggacttaatgattccttgattgacctcgaaacattgtcaattctcaaaacttatattaagtggccttggtatgtctggttagccatattcttcgccattattatattcatcctaatattaggatgggtctttttcatgactggttgttgtggttgttgttgcggatgctttggcattattccattaatgagcaagtgtggtaagaaatcttcctactacactacttttgataatgatgtggtaactgaacaatacagacctaaaaagtctgtttaatgatccaaagtcccactagtttcttaatagtattaattttgctttggtgtaaacttgtactaagttgttttagagagtttattattgcccttcaacaactaacacaagttttactccaaattatcgatagtaatttacagtctagactgaccctttggcacagtctagactaatgttaaacttagaagcaattattgaaaccggtgatcaagtgattcaaaaaatcagtttcaatttacagcatatttcaagtgtattaaacacagaagtatttgacccctttgactattgttattacagaggaggtaatttttgggaaatagagtcagctgaagattgttcaggtgatgatgaatttattgaataagtcgctagaggagaatggaagttttctaacggcactttacatatttgtaggatttttagcattttatcttctaggtagagcacttcaagcatttgtacaggctgctgatgcttgttgtttattttggtacacgtggttagtaattccaggagttaagggtacagcctttgtatacaagtatacatatggtagaaaacttaacaattcggaattagaagcagttgttgttaacgagtttcctaagaacggttggaataataaaaatccagcaaattttcaagatgtccaacgaaacaaattgtactcttgactttgaacagtcagttgagctttttaaagagtataatttatttataactgcattcttgttgttcttaaccataatacttcagtatggttatgcaacgcgtagtaagtttatttatatacttaaaatgatagtgttatggtgcttttggccccttaacattgcagtaggtgtaatttcatgtatatacccaccaaacacaggaggtcttgtcgcagcgataatacttactgtgtttgcgtgtctttcttttgtaggttattggatccagagtattagactctttaagcggtgtagatcttggtggtcatttaacccagaatctaacgccgtaggttcaatactcctaactaatggtcaacaatgtaattttgctatagagagtgtgccgatggtgctttctcctattataaagaatggtgttctttattgtgagggtcagtggcttgctaaatgtgaaccagaccacttgcctaaagacatatttgtatgcacaccagatagacgtaatatctatcgtatggtgcagaaatacactggtgaccaaagcggaaataagaaaaggtttgctacatttgtctatgcaaagcagtcagtagacactggcgagctagaaagtgtagcaacaggtggaagtagcctttacacataaatgtgtgtgtgtagagagtatttaaaattattcttcaatagtgcctctattttaagagcgcggaagagtatttgttttgaggatattaatataaatcctctttgttttgtactctctttacaagagttattatttaagcaacagtttttcctttcctttgtttggaagaaagttgttgttaatggtgtagaattccaagtagaaaatggaaaagtccactacgaaggaaaccccattttccaaaaaggttgttgtaggttgtggtcccattataagaaggattaaatggattaaaccacctacactacttacttgtaataagggcgtttggacttacaagcgcttaacaaatacagacgatgaaatggctgactagttttggaagagcagttatttcttgttataaagccctactattaactcagttaagagtattagataggttaattttagatcacggaccaaagcgagtcttaacgtgtggtaggcgagtgcttttatctcaattagatttagtttataggttggcatatacgcccacccaatcgctggtatgaataatagtaaagataatccttttcgcggagcaatagcaagaaaagcgcgaatttatctgagagaaggattagagtgtgtttactttcttaacaaagcaggacaagcagagccttgtcccgcgtgtacctccctagtatttcaggggaaaacttgtgaggaacacacagataataataatcttttgtcatggcgagcggtaagacaactgggaagacagacgccccagcgccagtcatcaaactaggagggccaaaaccacctaaagttggttcttctggaaatgctagctggtttcaagcactaaaagccaagaagttaaattcacctcctcctaagtttgaaggtagcggcgttcctgataatgaaaatcttaaattaagccagcaacatgggtactggagacgtcaagccaggtacaagccaggtaaaggcggaagaaaatcagtcccagatgcttggtacttctattacactggaacaggaccagccgctgacctgaattggggtgatagccaagatggtatagtgtgggtttctgcaaagggtgctgatactaaatctagatctaaccagggtacaagggatcctgataagtttgaccaatacccgctacgattctcagatggaggacctgatggtaatttccgttgggacttcattccaataaatcgtggtaggagtggaagatcaacagcggcttcatcagcagcatctagtagagcaccgtcgcgtgatggctcgcgtggacgtagaagcggagctgaagatgatcttatagctcgtgcagcaaagatcattcaggatcagcagaagaagggttctcgcattactaaagctaaggccgatgaaatggctcatcgccggtattgtaagcgtactatcccacctggttataaggttgatcaagtatttggtccccgtactaaaggtaaggagggaaattttggtgatgacaagatgaatgaggagggtattaaggatgggcgcgttacagcaatgctcaacctagtccctagcagccatgcttgtctttttggaagtagagtgacgcccaaacttcaaccagatgggctgcacttgagatttgaatttactactgtggtttctagggatgatccgcagtttgataattatgtgaaaatttgtgatcagtgtgtcgatggtgtagggactcggccaaaagacgatgaaccgagaccaaagtcacgcccaaattcaagacctgctacaagaacaagttctccagcgccaagacaacagcgtcaaaagaaggagaagaagtcaaagaagcaggatgatgaagtagataaggcattgacctcagatgaggagaggaacaatgcacagctggaatttgatgatgaaccgaaagtgattaactggggggattcagcacttggagagaatgagttgtaaagctagatttccaacttaacatcatggacgtgcgtatgctgtttttccctactatagactttttagcatattattttttgctatttgtatggtttattacaggtgaagattgtatgtatttgttgtacactcgtatgttctatattatgttttctgtagttgttattagtgttgttcttgttcttactctactgttctcttttctttattttagagtatcaataagaatcaaggaagataggcatgtagtttgattacctacatgtctatcgccagggaaatgtctaatctgtctacttagtagcctggaaacgaacggtagacccttagattttaatttagtttaatttttagtttagtttaagttagtttagagtaggtataaagaagccagtgccggggccacgcggagtacgatcgagggtacagcactaggacgcccactaggggaagagctaaattttagtttaagttaagtttaattggctaagtatagttaaaatttataggctagtatagagttagagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagtttaaactcggatccgcataggctgaggggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggaccctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtgacatggttcgtacaaaccaaatgcggtgttcttgtctggcaaagaggccaaggacgcagatggcaacaaacccaaggaacctgggataacggaggcttcatcggagataatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtctcctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaatagcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcact

SEQ ID NO: 15 - DNA, 20 bp, synthetic construct
  source 1..20: mol_type = other DNA
tgacttggtttgaagatggc

SEQ ID NO: 16 - DNA, 19 bp, synthetic construct
  source 1..19: mol_type = other DNA
agggctggtttgctcagac

SEQ ID NO: 17 - DNA, 18 bp, synthetic construct
  source 1..18: mol_type = other DNA
gcttttgagcctagcgtt

SEQ ID NO: 18 - DNA, 22 bp, synthetic construct
  source 1..22: mol_type = other DNA
gccatgttgtcactgtctattg

SEQ ID NO: 19 - DNA, 22 bp, synthetic construct
  source 1..22: mol_type = other DNA
caccaccagaacctgtcacctc

SEQ ID NO: 20 - protein, 1166 aa, Infectious bronchitis virus
MSVTRLLLVTLLFALCSAVLYDSNSYVYYYQSAFRPGSGWHLHGGAYAVVNVSEQTSPGSCTAGAIGYSMNFSAASVAMTAPLNGMSWSDRQFCMAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISARFPTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTDSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEYIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELLKWWNETKHDLPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 21 - protein, 1079 aa, Infectious bronchitis virus
VLYDSNSYVYYYQSAFRPGSGWHLHGGAYAVVNVSEQTSPGSCTAGAIGYSMNFSAASVAMTAPLNGMSWSDRQFCMAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISARFPTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTDSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEYIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELLKWWNETKHDLPDFDQFNYTIPVLNITYDIDKIEEVIKGLNDSLIDLETLSILKTYIKWP

SEQ ID NO: 22 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSGSQFCTAHCNFTNIVVFVTHCFKRGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSISSNVTNCPYVTYGRYCIEPDGSVKQIVPQELEQFVAPLLNVTEYVLIPNSFNLTVTDEYIQTRMDKVQINCLQYVCGNSLECRHLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 23 - protein, 1166 aa, Infectious bronchitis virus
MLVTRLLLVTLLFALCSAVLYDNNSYVYYYQSAFRPGSGWHLHGGAYAVVNVSAQTSPGRCTAGAIGYSMNFSAASVAMTAPLNGMSWSDSQFCTAHCNFTNIVVFVTHCFKSGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVSTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSISSNVTNCPYVTYGRYCIEPDGSVKQIVPQELEQFVAPLLNVTEYVLIPNSFNLTVTDEYIQTRMDKVQINCLQYVCGNSLECRHLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 24 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSDNQFCTAHCNFTNIVVFVTHCFKRGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVEYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSISSNVTNCPYVTYGRYCIEPDGSVKQIVPQELEQFVAPLLNVTEYVLIPNSFNLTVTDEYIQTRMDKVQINCLQYVCGNSLECRHLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 25 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSHNQFCTAHCNFTNIVVFVTHCFKRGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSISSNVTNCPYVTYGRYCIEPDGSVKQIVPQELEQFVAPLLNVTEYVLIPNSFNLTVTDEYIQTRMDKVQINCLQYVCGNSLECRHLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 26 - protein, 1166 aa, Infectious bronchitis virus
MLVTRLLLVTLLFALCSAVLYDNNSYVYYYQSAFRPGSGWHLHGGAYAVVNVSAQTSPGRCTAGAIGYSMNFSAASVAMTAPLNGMSWSDSQFCTAHCNFTNIVVFVTHCFKSGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSISSNVTNCPYVTYGRYCIEPDGSVKQIVPQELEQFVAPLLNVTEYVLIPNSFNLTVTDEYIQTRMDKVQINCLQYVCGNSLECRHLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 27 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSDRQFCTAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTGSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRNLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEHIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELSKWWNETKHDLPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 28 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSDRQFCTAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNVTGSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRNLFQQYGPVCDNILSVVNSVGQKEDMELLSFYSSTKPTGYNTPIFNNLTTGAFNVSLLLPQPGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEYIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELSKWWNETKHDLPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 29 - protein, 1166 aa, Infectious bronchitis virus
MLVTRLLLVTLLFALCSAVLYDNNSYVYYYQSAFRPGSGWHLHGGAYAVVNVSAQTSPGSCTAGAIGYSMNFSAASVAMTAPLNGMSWSNSQFCTAHCNFTNIVVFVTHCFKRGQQFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAQDVILCDGTPRGLLACQYNTGNFTDGFYPFTNSSLVKERFIVYRENSVNTTLVLHNFTFFNETSAPPAGGDISATFQTYQTVTAQSGYYNFNFSFLSGFVYRASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGVLTSRNVTGSQFLENQFYIKLTNETHRSRRSVSENVTSCPYVSYGKFCIKPDGSISVIVPKELEQFVAPLLNVTEYVLIPDSFNLTVTDEYIQTRMDKVQINCLQYVCGNSLECRKLFQQYGPVCDNILSVVNSVGQKEDMELLSFYSSTKPTGYNTPIFNNLTTGAFNVSLLLPQPGSPTGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGQMQEGFRSTSLALQQIQDVVNKQSAILTETMASLNKNFGAISSVIQDIYQQLDVIQADAQVDRLITGRLSSLSVLASAKQSEHIIASQQRELATQKINECVKSQSTRYSFCGNGRHVLTIPQNAPNGIVFIHFTYTPESFVNVTAIVGFCVNPANASQYAIVPANGRGIFIQVNGTYYITARDMYMPRSITAGDIVTLTSCQANYVSVNKTVITTFVEDDDFNFDDELSKWWNETKHDLPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 30 - protein, 1166 aa, Infectious bronchitis virus; identical to SEQ ID NO: 29

SEQ ID NO: 31 - protein, 1166 aa, Infectious bronchitis virus; identical to SEQ ID NO: 29

SEQ ID NO: 32 - protein, 1166 aa, Infectious bronchitis virus
MSVTRLLLVTLLFALCSAVLYDNNSYVYYYRSAFRPGSGWHLHGGAYAVVNVSAQTSPGSCTAGAIGYSMNFSAASVAMTAPLNGMSWSDSQFCTAHCNFTNIVVFVTHCFKRGQHFCPLTGFIEQNYIRIAAMRINGTGPRDLFYNSTVSVSKYPKFRSLQCVNNQTSVYLNGDLVFTSNETLYVSGAGVDFTAGGPITYKVMQEVKALAYFVNGTAHDVILCDGTPRGLLACQYNTGNFTDGFYPFTNSSLVKERFIVYRENSVNTILVLHNFTFFNETSAPPAGGDISATFQTYQTVTAQSGYYNFNFSFLSGFVYKASDFMYGSYHPKCNFRPETLNNDLWFNSLSISLAYGPLQGGCKQSVFNNRATCCYAYSYNGPRACKGVYRGQLQQLFECGLLVYITKSDGSRIQTTTKPHVLTTHFYNNITLDKCVDYNIYGRVGQGFITNVTDSTAAYNYLADGGLAILDTSGAIDIFVVQGEYGLNFYKVNPCEDVNQQFVVSGGKLVGILTSRNETDSHHLEGQFYIKLTNGSRRSRRSIISNVTNCPYVTYGRYCIEPDGSVKQIVPQELEQFVAPLLNVTEYVLIPNSFNLTVTDEYIQTRMDKVQINCLQYVCGNSLECRQLFQQYGPVCDNILSVVNSVGQKEDMELLSFYSSTKPTGYNTPIFNNLTTGAFNVSLLLPQSGSPRGRSFVEDLLFTSVETVGLPTDAEYKKCTAGPLGTLKDLICAREYNGLLVLPPIITADMQTMYTASLVGAMAFGGITSAAAIPFATQIQARINHLGITQSLLMKNQEKIAASFNKAIGHMQEGFRSTSLALQQIQDVVNKQSAILTETMQSLNKNFGAISSVIQDIYQQLDSIQADAQVDRLITGRLSSLSVLASAKQSEYLRVSQQRELATQKINECVKSQSSRYSFCGNGRHVLTIPQNAPNGIVFIHFTYTPESFVNVTAIVGFCVTPANASQYAIVPANGRGIFIQVNGSYHITARDMYMPRDITAGDIVTLTSCQANYVSVNKTVITTFVEDDDFDFDDELSKWWNDTKHELPDFDQFNYTIPVLNITYDIDKIEEVIKGLNDSLIDLETLSILKTYIKWPWYVWLAIFFAIIIFILILGWVFFMTGCCGCCCGCFGIIPLMSKCGKKSSYYTTFDNDVVTEQYRPKKSV

(19) United States
(12) Patent Application Publication
(10) Pub. No.: US 2025/0064920 A1
(43) Pub. Date: Feb. 27, 2025

(54) IBV VACCINE WITH HETEROLOGOUS DMV/1639 SPIKE PROTEIN

(71) Applicant: Boehringer Ingelheim Vetmedica GmbH, Ingelheim am Rhein (DE)

(72) Inventors: Grace Albanese, Athens, GA (US); Aemro KASSA, Watkinsville, GA (US); Annika KRAEMER-KUEHL, Seesen (DE); Stephane LEMIERE, Lyon (FR)

(21) Appl. No.: 18/790,098

(22) Filed: Jul. 31, 2024

Related U.S. Application Data
(60) Provisional application No. 63/517,502, filed on Aug. 3, 2023.

(51) Int. Cl. A61K 39/215; A61K 9/00; A61K 39/00; A61P 31/14; C07K 14/005; C12N 7/00

(52) U.S. Cl. CPC A61K 39/215; A61K 9/0073; A61P 31/14; C07K 14/005; C12N 7/00; A61K 2039/544; A61K 2039/552; C12N 2770/20022; C12N 2770/20034

(57) ABSTRACT

The present invention relates i.a. to an IBV (infectious bronchitis virus) encoding for a heterologous DMV S (spike) protein or fragment thereof. Further, the present invention relates to an immunogenic composition comprising said IBV encoding for a heterologous DMV S (spike) protein or fragment thereof. Furthermore, the present invention relates to methods for immunizing a subject comprising administering to such subject the immunogenic composition of the present invention. Moreover, the present invention relates to methods of treating or preventing clinical signs caused by IBV in a subject of need, the method comprising administering to the subject a therapeutically effective amount of an immunogenic composition according to the present invention.

### SEQUENCE LISTING

This application contains a sequence listing in accordance with 37 C.F.R. 1.821-1.825. The sequence listing accompanying this application is hereby incorporated by reference in its entirety.

### BACKGROUND OF THE INVENTION

Avian infectious bronchitis virus (IBV) is a gammacoronavirus with a genome about 27.6 kb in size. Infection of chickens with IBV causes the disease infectious bronchitis (IB). IB was first identified in 1931, and it is still a major cause of production losses in the global poultry industry today (Jackwood et al 2021: Avian Dis 65, 631-636). Clinical signs for IB include nasal discharge, snicking, respiratory rales, watery eyes, dyspnea, depression, and swollen sinuses (Swayne et al 2013: Diseases of Poultry, Wiley-Blackwell). IBV infects epithelial tissues, and infection typically results in respiratory tract disease. Variant strains of IBV can cause increased disease in non-respiratory epithelial tissues, including the reproductive tract and kidneys (Hassan et al 2021: Viruses 13, 2488). Although mortality from IB is typically low, disease incidence results in major economic losses for the commercial poultry industry due to reduced weight gain, production condemnations due to secondary bacterial infection, and decreased egg production and quality (Jordan et al 2017: Vet Microbiol 206, 137-143).

The genome organization of IBV is 5′UTR-ORF1a-ORF1b-S-3a-3b-E(3c)-M-5a-5b-N-3′UTR. The 5′ two-thirds of the genome code for the replicase polyproteins 1a and lab. Polyproteins 1a and lab are proteolytically cleaved into 15 non-structural proteins (nsp2-nsp16). These non-structural proteins have roles in pathogenicity, attenuation, replication, and code for the viral RdRp (Jackwood et al 2021: Avian Dis 65, 631-636). The structural proteins S, E, M, and N are produced from a set of 3′ nested subgenomic mRNAs, which is a characteristic feature of viruses in the Nidovirales order (Enjuanes et al. 2008: Encycl Virology 419-430, Grellet et al 2022: J Biological Chem 101923). The S gene codes for the spike glycoprotein, which is the major antigen of IBV (Kant et al 1992: J Gen Virol 73, 591-596, Moore et al 1997: Arch Virol 142, 2249-2256). The spike glycoprotein is responsible for attachment to the host cell and is the determinant of tissue tropism of IBV. Spike is post-translationally proteolytically cleaved into S1 and S2 subunits (Makhija et al 2015: Can J Microbiol 61, 983-989). The S1 subunit contains domains for the receptor binding domain of IBV which targets host sialic acid receptors (Schultze et al 1992: Virology 189, 792-794), while the S2 subunit is responsible for fusion to the endosome upon viral entry into the host cell (Hulswit et al 2016: Adv Virus Res 96, 29-57, Andoh et al 2018: Avian Dis 62, 210-217). Although both S1 and S2 have novel epitopes, S1 is the site for binding of neutralizing antibodies (Moore et al 1997: Arch Virol 142, 2249-2256, Stevenson-Leggett et al 2021: J Gen Virology 102, 001642). The mature IBV spike glycoprotein is a trimer, with each trimeric subunit consisting of an S1 head and an S2 stalk (Shang et al 2018: Plos Pathog 14, e1007009).

Protection against IB is achieved using attenuated modified live vaccines (MLV) and inactivated vaccines. Modified live vaccines are attenuated via serial passage of a pathogenic IBV field isolate in embryonated chicken eggs (Cavanagh 2007: Vet Res 38, 281-297). After enough passages (typically 100 or more), the virus adapts to tropism for embryo infection and replication, which reduces virulence in chicken tissues (Britton et al 2012: Bioengineered 3, 114-119). To date most widely used live-attenuated IBV vaccine strains were developed in the 1960s in the Netherlands, by serial passaging of a Massachusetts-like IBV strain. However, since the 1970s new IBV serotypes emerged against which the traditional Massachusetts-like vaccines did not protect sufficiently (Cook et al. 2012. Avian Pathol. 41:239-250). Therefore, there is a need for new and highly efficacious IBV vaccines against other IBV serotypes.

The IBV variant strain DMV/1639 has been causing significant economic losses in the North American poultry industry since the mid-2010's. This strain was originally isolated on the Delaware, Maryland, Virginia (DMV) peninsula in the US and originally caused severe nephropathogenic disease in broiler chickens. In 2017, disease from DMV/1639 infection became more severe in laying chickens, in particular laying flocks in Canada (Hassan et al 2019: Viruses 11, 1054). False layer syndrome, in which mature layer hens never come into production due to cystic oviducts, was linked to DMV/1639 infection (Parent et al 2020: Avian Dis 64, 149-156). This was detected in pullet flocks in the US as well. DMV/1639 detection in broiler chickens increased again in 2019, and to date the DMV/1639 variant strain has been detected in nearly all commercial poultry producing regions in the US (Jackwood et al 2021: Avian Dis 65, 631-636).

Currently, the only commercially licensed vaccine available with a protection claim against DMV/1639 is Cevac IBron®, which contains the Georgia (GA) serotype of IBV and only has a cross-protection claim against DMV/1639 and only when applied via gel droplet by oral administration. However, the Cevac IBron® does not comply with the 90% protection requirement for efficacy as described in the US Title 9 Code of Federal Regulations, section 113.327 when applied via coarse spray as published in the Cevac IBron®LYO product sheet. For that reason Cevac IBron® has not been licensed against DMV/1639 by coarse spray vaccination. This is a disadvantage since the most commonly used method for mass vaccination against IBV is coarse spray. The advantage of using coarse spray is that coarse spray vaccination cabinets are more frequently used (are more frequently available) in chicken hatcheries.

WO22066683 discloses a heat attenuated DMV/1639 IBV isolate. For the efficacy test, only viral loads by qPCR post-challenge have been evaluated. However, virus isolation in chicken embryos is the gold standard for detection of IBV (Banda et al 2022: Springer Protocols Handbooks, 115-26). Virus isolation in embryos is also the assay codified for evaluation of IBV vaccine efficacy by the US Title 9 Code of Federal Regulations Section 113.327. The 9-CFR testing of virus isolation in embryos has not been done and, therefore, their protection rates based on qPCR data can not be compared to the codified test via virus isolation.

All IBV spike sequences used in any vector approach so far vary significantly from any DMV/1639 spike sequence. This may be the reason why no recombinant or vector based approach has been described or suggested so far using a DMV/1639 specific spike. Due to the sequence variation, no reasonable assumption can be made whether such vector based approach using any DMV/1639 specific spike sequence will work and will provide sufficient protection after DMV/1639 infection.

Consequently, there is a need for generating a novel, highly efficacious rIBV vaccine to provide homologous protection against the DMV/1639 variant strain.

### DETAILED DESCRIPTION OF THE INVENTION

Before the aspects of the present invention are described, it must be noted that as used herein and in the appended claims, the singular forms “a”, “an”, and “the” include plural reference unless the context clearly dictates otherwise. Thus, for example, reference to “an antigen” includes a plurality of antigens, reference to the “virus” is a reference to one or more viruses and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies as reported in the publications which might be used in connection with the invention. Nothing herein is to be construed as an admission that the invention is not entitled to antedate such disclosure by virtue of prior invention.

### Composition of Matter

The present invention solves the problems inherent in the prior art and provides a distinct advance in the state of the art.

Generally, the present invention provides an IBV (infectious bronchitis virus) encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.

Advantageously, the IBV or immunogenic composition as provided herein has been proven to be efficacious in reducing the viral load after infection/challenge. The IBV or immunogenic composition as provided herein confers 90% protection against IBV DMV/1639 homologous challenge. This was not expected since the DMV/1639 spike sequence varies more than 15% from the QX, CR88 and the other spike sequences used so far in this vector approach.

The term “IBV” refers to an infectious bronchitis virus. IBV strains are typically differentiated by the coding sequence of the S1 subunit of the spike protein (Valastro et al. 2016. Infect Genet Evol. 39:349-364) but can also be differentiated by their complete nucleotide sequence or the sequences of specific proteins such as the spike protein (S), Nucleocapsid (N) protein, envelope (E) protein or membrane (M) glycoprotein. Because the spike protein determines host tropism and antigenicity of IBV, the IBV genotypes are classified by the coding sequence of the subunit 1 of the spike proteins. Alternatively, IBV strains can be differentiated by their serotype. Serotype classification involves treatment of the virus with neutralizing antibodies.

The term “spike” refers to a specific protein of the IBV that is well known by the person skilled in the art. The spike protein is the major inducer of antibodies and protective immune response. Further, the spike (S) protein facilitates cell entry of IBV by binding cellular receptors of the host cell and also by mediating virus-cell membrane fusion with the host cell. In addition, it determines the tissue and cell tropism of the virus strain.

The term “heterologous S (spike)” means that the spike protein or fragment thereof that has been introduced into the IBV is different compared to its natural context. Accordingly, such IBV encoding for a heterologous IBV spike protein or fragment thereof is a recombinant IBV. The term “recombinant” has been defined elsewhere. The heterologous spike protein is from a DMV/1639 IBV. Accordingly, the IBV (IBV backbone) is not derived from DMV/1639.

The term “protein”, “amino acid” and “polypeptide” are used interchangeably. The term “protein” refers to a sequence of amino acids composed of the naturally occurring amino acids as well as derivatives thereof. The naturally occurring amino acids are well known in the art and are described in standard text books of biochemistry. Within the amino acid sequence the amino acids are connected by peptide bonds. Further, the two ends of the amino acid sequence are referred to as the carboxyl terminus (C-terminus) and the amino terminus (N-terminus). The term “protein” encompasses essentially purified proteins or protein preparations comprising other proteins in addition. Further, the term also relates to protein fragments. Moreover, it includes chemically modified proteins. Such modifications may be artificial modifications or naturally occurring modifications such as phosphorylation, glycosylation, myristylation and the like.

The term “identity” or “sequence identity” is known in the art and refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

The terms “identity”, “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are of the same length.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

The skilled person will be aware of the fact that different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

The protein sequences or nucleic acid sequences of the present invention can further be used as a “query sequence” to perform a search against public databases to, for example, to identify other family members or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

As used herein, it is in particular understood that the term “identical to the sequence of SEQ ID NO: X” is equivalent to the term “identical to the sequence of SEQ ID NO: X over the length of SEQ ID NO: X” or to the term “identical to the sequence of SEQ ID NO: X over the whole length of SEQ ID NO: X”, respectively. In this context, “X” is any integer selected from 1 to 32 so that “SEQ ID NO: X” represents any of the SEQ ID NOs mentioned herein.

Generally, the present invention provides an H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof. In one aspect, the H52 IBV has or comprises the sequence as shown in SEQ ID NO: 3.

The term “H52 IBV” is well known to the person skilled in the art. The term “H52” defines the specific IBV strain. The H52 strain is well known to the person skilled in the art and belongs to the Massachusetts serotype, genotype GI-1.

Further, H52 can be differentiated from H120 by higher pathogenicity upon application in young chickens.

It is in the general knowledge of a person skilled in the art where to obtain H52 IBV. H52 IBV strains can be commercially purchased such as exemplary Nobilis IB H52 (MSD Animal Health), AviPro IB H52 (Lohmann Animal Health GmbH & Co. KG), Bronchovac (Ceva) and the alike. Further, McDonald et al. 1980 (Avain Pathology 9:245-259) disclose that H52 IBV can be obtained by Central Veterinary Laboratory Rotterdam. Kusters et al. 1987 (J. gen Virol 68:343-352) disclose that H52 IBV can be obtained by the Poultry Health Institute Dorn in the Netherlands (which is now GD Animal Health) and Chen et al. 2007 (Avian Pathology 36(4):269-274) disclose that H52 IBV can be obtained by the China Institute of Veterinary Drug Control. Furthermore, H52 IBV is used as vaccine strain for decades (Bijlenga et al. 2004, Avian Pathology 33 (6): 550-557) and, therefore, can be isolated from the field. The methods to isolate H52 IBV strains and to characterize the H52 IBV strains are well known to the person skilled in the art. Exemplary, H52 IBV strains can be characterized as described in Zwaagstra et al. 1992 (J. Clin. Microbiol. 30 (1): 79-84), Handberg et al. 1999 (Avian Pathology 28: 327-335) or Callison et al. 2006 (Journal of Virological Methods 138: 60-65). Zwaagstra et al. 1992 and Handberg et al. 1999 for example disclose Massachusetts specific primers (for the S and N protein, respectively) for RT-PCR and sequencing and reference sequences for comparison. Further, H52 IBVs have been sequenced and the genomic sequences are available such as EU817497. Thus, the virus genome can be generated by synthesizing its sequence and generated upon the application of reverse genetic systems.

The term “heterologous S (spike)” means that the spike protein or fragment thereof that has been introduced into the H52 IBV is from a different genotype or serotype than the H52 IBV. Because the IBV is a H52 IBV and the heterologous spike protein is from a DMV/1639 IBV, the spike protein is heterologous.

Further, the present invention also provides a H52 IBV (infectious bronchitis virus) encoding for a heterologous DMV/1639 IBV spike protein or fragment thereof.

The term “DMV/1639” is well known to the person skilled in the art. DMV/1639 stands for Delmarva/1639. DMV/1639 belongs to the GI-17 genotype.

It is in the general knowledge of a person skilled in the art how and where to obtain DMV/1639 strains and spike sequences. DMV/1639 strains and spike proteins have been described exemplarily in Goraichuk et al. 2019 (Microbiol Resour Announc.: 8(34): e00840-19), Jackwood et al. 2017 (PLoS One 4; 12(5):e0176709), Hassan et al. 2019 (Viruses:11, 1054) and Silva et al. 2022 (Viruses: 14, 1998) with its isolate names and some accession numbers. Accession numbers disclosing DMV/1639 specific spike proteins are such as QCX19619 (SEQ ID NO: 20), QGM12378 (SEQ ID NO: 22), QGM12391 (SEQ ID NO: 23), QGM12404 (SEQ ID NO: 24), QGM12416 (SEQ ID NO: 25), QGM12429 (SEQ ID NO: 26), QRG28806 (SEQ ID NO: 27), QRG28819 (SEQ ID NO: 28), UXN85484 (SEQ ID NO: 29), UXN85494 (SEQ ID NO: 30), UXN85504 (SEQ ID NO: 31) and UZP65106 (SEQ ID NO: 32). Therefore, DMV/1639 spike sequences are publicly available and the spike sequences can be synthetically synthesized de novo. Further, the genotyping of DMV/1639 has been described as well, exemplarily in Ali et al. 2022 (Genes: 13(9):1617) and Hassan et al. 2019 (Viruses 11,1054.), therefore, DMV/1639 strains can be isolated from the field as well.

Further, the present invention also provides an immunogenic composition comprising a H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or a fragment thereof. In one aspect, the H52 IBV has or comprises the sequence as shown in SEQ ID NO: 3.

The term “immunogenic composition” refers to a composition that comprises at least one antigen, which elicits an immunological response in the host to which the immunogenic composition is administered. Such immunological response may be a cellular and/or antibody-mediated immune response to the immunogenic composition of the invention. Preferably, the immunogenic composition induces an immune response and, more preferably, confers protective immunity against one or more of the clinical signs of a IBV infection. The host is also described as “subject”. Preferably, any of the hosts or subjects described or mentioned herein is an avian or poultry.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutical response.

A “protective immunological response” or “protective immunity” will be demonstrated by either a reduction or lack of clinical signs normally displayed by an infected host, a quicker recovery time and/or a lowered duration of infectivity or lowered pathogen titer in the tissues or body fluids or excretions of the infected host.

In case where the host displays a protective immunological response such that resistance to new infection will be enhanced and/or the clinical severity of the disease reduced, the immunogenic composition is described as a “vaccine”.

Furthermore, the present invention also provides an immunogenic composition comprising an IBV (infectious bronchitis virus) as described herein. Thus, provided is an immunogenic composition comprising an IBV (infectious bronchitis virus) encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof. Further, provided is an immunogenic composition comprising a H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof. In one aspect, the H52 IBV has or comprises the sequence as shown in SEQ ID NO: 3. Furthermore, provided is an immunogenic composition comprising a H52 IBV (infectious bronchitis virus) encoding for a heterologous DMV/1639 IBV spike protein or fragment thereof.

### IBV Vector Definition

In one specific aspect of the IBV or the immunogenic composition according to the present invention the IBV is selected from the group consisting of: H52, H120, 4/91 and QX.

H52 as a vector backbone has already been described in the prior art, exemplarily in WO2020/089166. Further, 4/91 as a vector backbone has already been described in the prior art, exemplarily in WO2020/089164. Furthermore, QX as a vector backbone has already been described in the prior art as well (Zhao et al 2019: Virus Research 272:197726; Zhao et al 2019: Veterinary Microbiology: 239:108464).

In one specific aspect of the IBV or the immunogenic composition according to the present invention the IBV is H52 or H120.

### H52-IBV—Definition by Protein Encoding Sequences

In another specific aspect of the IBV, H52 IBV or the immunogenic composition according to the present invention the H52 IBV has or consists of or comprises a nucleotide sequence as shown for EU817497 (SEQ ID NO:3) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

The term “nucleic acid” or “nucleic acid sequence” or “nucleotide sequence” refers to polynucleotides including DNA molecules, RNA molecules, cDNA molecules or derivatives. The term encompasses single as well as double stranded polynucleotides. The nucleic acid of the present invention encompasses isolated polynucleotides (i.e. isolated from its natural context) and genetically modified forms. Moreover, comprised are also chemically modified polynucleotides including naturally occurring modified polynucleotides such as glycosylated or methylated polynucleotides or artificially modified ones such as biotinylated polynucleotides. Further, the terms “nucleic acid” and “polynucleotide” are interchangeable and refer to any nucleic acid. The terms “nucleic acid” and “polynucleotide” also specifically include nucleic acids composed of bases other than the five biologically occurring bases (adenine, guanine, thymine, cytosine and uracil).

The term “RNA” refers to any ribonucleic acid. The term encompasses single as well as double stranded RNAs. The RNA of the present invention encompasses isolated RNA (i.e. isolated from its natural context) and genetically modified forms. Moreover, comprised are also chemically modified RNAs including naturally occurring modified RNAs such as methylated RNA or artificially modified ones such as biotinylated RNA. The terms “RNA” also specifically include RNA composed of bases other than the four biologically occurring nucleotides/bases (adenine, guanine, cytosine and uracil).

In another specific aspect of the IBV, H52 IBV or the immunogenic composition according to the present invention the H52 IBV strain has or consists of or comprises a spike (S) protein having the amino acid sequence as shown for AAK27168 (SEQ ID NO:4) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

It has to be understood that the spike protein or nucleic acid sequence can be used to determine whether any IBV strain is of H52 origin. However, because the H52 IBV is used as a backbone and the H52 spike protein or nucleic acid sequence is replaced by a heterologous spike or fragment thereof, the final IBV with the heterologous spike does not comprise any or only remaining parts of the H52 spike.

In another specific aspect of the IBV, H52 IBV or the immunogenic composition according to the present invention the H52 IBV strain has or consists of or comprises a spike (S) protein having the amino acid sequence as shown SEQ ID NO:5 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has or consists of or comprises a nucleocapsid (N) protein having the amino acid sequence as shown for AAK91809 (SEQ ID NO:6) or AAK27163 (SEQ ID NO:7) a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has or consists of or comprises a nucleocapsid (N) protein having the amino acid sequence as shown SEQ ID NO:8 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has or consists of or comprises an envelope (E) protein having the amino acid sequence as shown for AAL26888 (SEQ ID NO:9) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has or consists of or comprises an envelope (E) protein having the amino acid sequence as shown in SEQ ID NO:10 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has or consists of or comprises a membrane glycoprotein (M) protein having the amino acid sequence as shown for AAK83031 (SEQ ID NO:11) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the H52 IBV has a or consists of or comprises membrane glycoprotein (M) protein having the amino acid sequence as shown in SEQ ID NO:12 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.

### DMV/1639 S Protein

DMV/1639 strains and spike proteins have been described exemplarily in Goraichuk et al. 2019 (Microbiol Resour Announc.: 8(34): e00840-19) and Jackwood et al. 2017 (PLoS One 4; 12(5):e0176709) with its accession numbers. Therefore, DMV/1639 spike sequences are publicly available and the spike sequences can be synthetical synthesized de novo. Further, the genotyping of DMV/1639 has been described as well, exemplarily in Ali et al. 2022 (Genes: 13(9):1617) and Hassan et al. 2019 (Viruses 13; 11(11):1054.), therefore, DMV/1639 strains can be isolated from the field as well.

In another specific aspect of the IBV, H52 IBV or the immunogenic composition according to the present invention the DMV/1639 spike consists of or comprises a sequence selected from the list consisting of: SEQ ID NO: 20 (QCX19619), SEQ ID NO: 22 (QGM12378), SEQ ID NO: 23 (QGM12391), SEQ ID NO: 24 (QGM12404), SEQ ID NO: 25 (QGM12416), SEQ ID NO: 26 (QGM12429), SEQ ID NO: 27 (QRG28806), SEQ ID NO: 28 (QRG28819), SEQ ID NO: 29 (UXN85484), SEQ ID NO: 30 (UXN85494), SEQ ID NO: 31 (UXN85504) and SEQ ID NO: 32 (UZP65106) or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the DMV/1639 spike consists of or comprises a sequence selected from the list consisting of: SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the DMV/1639 spike consists of or comprises a sequence selected from the list consisting of: SEQ ID NO: 20 (QCX19619), SEQ ID NO: 22 (QGM12378), SEQ ID NO: 23 (QGM12391), SEQ ID NO: 24 (QGM12404), SEQ ID NO: 25 (QGM12416), SEQ ID NO: 26 (QGM12429), SEQ ID NO: 27 (QRG28806), SEQ ID NO: 28 (QRG28819), SEQ ID NO: 29 (UXN85484), SEQ ID NO: 30 (UXN85494), SEQ ID NO: 31 (UXN85504) and SEQ ID NO: 32 (UZP65106).

In another specific aspect of the IBV, H52 IBV or the immunogenic composition according to the present invention the DMV/1639 spike consists of or comprises the sequence as shown in SEQ ID NO: 20 (QCX19619) or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the DMV/1639 spike is from the DMV accession number QCX19619.

In another specific aspect of the IBV, H52 IBV or the immunogenic composition according to the present invention the heterologous DMV/1639 S protein has at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 1, 2, 20 or 21 or the heterologous DMV/1639 S protein consists of or comprises the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.

In another specific aspect of the IBV, H52 IBV or the immunogenic composition according to the present invention the heterologous DMV/1639 S protein is selected from a list consisting of SEQ ID NO: 1, 2, 20 or 21.

In another specific aspect of the IBV, H52 IBV or the immunogenic composition according to the present invention the heterologous DMV/1639 S protein has one or more amino acid(s) selected from the list consisting of: 242T, 489F and 1038L.

The positions 242T, 489F and 1038L are only found in the DMV/1639 S protein, but not in any other IBV spike proteins.

In another specific aspect of the IBV, H52 IBV or the immunogenic composition according to the present invention the heterologous DMV/1639 S protein has the amino acid 242T and/or 489F and/or 1038L. Thus, the DMV/1639 S protein may have the amino acid 242T or 489F or 1038L or 242T and 489F or 242T and 1038L or 489F and 1038L or 242T and 489F and 1038L.

In another specific aspect of the IBV, H52 IBV or the immunogenic composition according to the present invention the heterologous DMV/1639 S protein has the amino acid 242T and 489F and 1038L.

In another specific aspect of the IBV, H52 IBV or the immunogenic composition according to the present invention the numbering of the amino acid positions refer to the amino acid positions in a DMV/1639 spike protein.

In another specific aspect of the IBV, H52 IBV or the immunogenic composition according to the present invention the numbering of the amino acid positions 242T, 489F and 1038L refer to the amino acid positions in a DMV/1639 spike protein.

### Extended Cell or Tissue Tropism

In another specific aspect of the IBV, H52 IBV or the immunogenic composition according to the present invention the heterologous DMV/1639 S protein has the amino acid Cysteine at amino acid position 270 or the heterologous DMV/1639 S protein has the amino acid 270C.

In another specific aspect of the IBV, H52 IBV or the immunogenic composition according to the present invention the heterologous DMV/1639 S protein has the amino acid L270C.

In another specific aspect of the IBV, H52 IBV or the immunogenic composition according to the present invention the Cysteine at amino acid position 270 is introduced by a mutation.

In another specific aspect of the IBV, H52 IBV or the immunogenic composition according to the present invention the Cysteine at amino acid position 270 leads to an extended cell or tissue tropism of the IBV.

The term “cell or tissue” is known by the person skilled in the art. The term cell encompasses cell lines such as the cell lines listed elsewhere herein as well as primary cells. The term tissue encompasses cells from tissues exemplarily such as primary chicken embryo cells from lung or liver or primary chicken fibroblasts. The term encompasses the propagation of cells or tissue (cells) in culture outside the organism. The term “culture” relates to the propagation of cells (such as cell line cells or primary cells or tissue cells) outside the organism under defined culture conditions known by the person skilled in the art.

The term “extended tropism” means that the IBV can be propagated in cells (such as cell lines) or tissue cells (in addition to primary chicken embryo cells from kidney). In contrast, IBV vaccines or non-cell culture adapted wildtype IBV's (cell line adapted IBV Beaudette strains are described) can only be propagated in embryonated chicken eggs or primary chicken embryo cells from kidney (after adaption). Accordingly, an IBV with extended cell or tissue tropism has the capacity to infect and/or replicate in one or more cell lines or tissue cells other than primary chicken embryo cells from kidney. Accordingly, an IBV with extended cell or tissue tropism may, for example, have the capacity to infect and/or replicate in PBS-12SF, EB66 or HEK 293T cells.

WO 2020/229248 A already describes that said single amino acid modification within the spike protein leads to an extended cell culture tropism. WO 2020/229248 gives further guidance on how to introduce such mutation and the cell lines that can be used for cultivation.

### Numbering of the Amino Acid Positions

In another specific aspect of the IBV, H52 IBV or the immunogenic composition according to the present invention the numbering of amino acid positions refer to the amino acid positions in the spike protein as given in SEQ ID NO:1.

In another specific aspect of the IBV, H52 IBV or the immunogenic composition according to the present invention the numbering of amino acid positions 242T, 270C, 489F and 1038L refer to the amino acid positions 242T, 270C, 489F and 1038L in the spike protein as given in SEQ ID NO:1.

In another specific aspect of the IBV, H52 IBV or the immunogenic composition according to the present invention the amino acid sequence of SEQ ID NO:1 is used for determining the position numbering in the spike protein.

In another specific aspect of the IBV, H52 IBV or the immunogenic composition according to the present invention the amino acid sequence of SEQ ID NO:1 is used for determining the position numbering of 242T, 270C, 489F and 1038L in the spike protein.

In another specific aspect of the IBV, H52 IBV or the immunogenic composition according to the present invention for determining the amino positions in a spike protein the amino acid sequence is aligned to the amino acid sequence of SEQ ID NO:1.

In another specific aspect of the IBV, H52 IBV or the immunogenic composition according to the present invention for determining the amino positions 242T, 270C, 489F and 1038L in a spike protein the amino acid sequence is aligned to the amino acid sequence of SEQ ID NO:1.

In another specific aspect of the IBV, H52 IBV or the immunogenic composition according to the present invention the amino acid positions 242T, 270C and 489F are within the S1 subunit of the spike protein and 1038L is within the S2 subunit of the spike protein.

In another specific aspect of the IBV, H52 IBV or the immunogenic composition according to the present invention the heterologous S protein is the full length spike protein.

### Fragments

In another specific aspect of the IBV, H52 IBV or the immunogenic composition according to the present invention the fragment of the heterologous DMV/1639 S (spike) protein has a length of at least 1000, at least 1050 or at least 1077 amino acids.

In another specific aspect of the IBV, H52 IBV or the immunogenic composition according to the present invention the fragment of the heterologous DMV/1639 S (spike) protein has a length of at least 1000, at least 1050 or 1077 amino acids from the N-Terminus.

The term “N-terminus” is well known to the person skilled in the art. The N-terminus is also termed amino-terminus, NH2-terminus, N-terminal end or amine-terminus. When the protein is translated from messenger RNA, it is created from N-terminus to C-terminus. Thus, the N-terminus is the start of an amino acid chain (protein or polypeptide) comprising said amine group (—NH2).

In another specific aspect of the IBV, H52 IBV or the immunogenic composition according to the present invention the fragment of the heterologous DMV/1639 S (spike) protein has a length of at least 1000 amino acids.

In another specific aspect of the IBV, H52 IBV or the immunogenic composition according to the present invention the fragment of the heterologous S (spike) protein is the ectodomain of the spike protein.

The term “ectodomain” is well known to a person skilled in the art. The spike protein comprises different functional parts, the signal sequence, the ectodomain, the transmembrane domain and the endodomain (from N-terminus to C-terminus). Thus, after cleavage of the signal sequence, the N-terminus of the spike protein starts with the ectodoamain. The IBV spike ectodoamins has a length of about 1077 amino acids and differs by a a few amino acids in length dependent on the IBV strain.

In another specific aspect of the IBV, H52 IBV or the immunogenic composition according to the present invention the heterologous DMV S (spike) protein replaces the homologous S protein.

In another specific aspect of the IBV, H52 IBV or the immunogenic composition according to the present invention the heterologous DMV/1639 S (spike) protein replaces the natural occurring S protein.

In another specific aspect of the IBV, H52 IBV or the immunogenic composition according to the present invention the heterologous S (spike) protein replaces the S protein in H52, H120, 4/91 or QX.

In another specific aspect of the IBV, H52 IBV or the immunogenic composition according to the present invention the heterologous S (spike) protein replaces the S protein in H52.

In another specific aspect of the IBV, H52 IBV or the immunogenic composition according to the present invention the IBV is attenuated.

The term “attenuated” refers to a pathogen having a reduced virulence in comparison to the wildtype isolate. In the present invention, an attenuated IBV is one in which the virulence has been reduced so that it does not cause clinical signs of an IBV infection but is capable of inducing an immune response in the target animal, but may also mean that the clinical signs are reduced in incidence or severity in animals infected with the attenuated IBV in comparison with a “control group” of animals infected with non-attenuated IBV and not receiving the attenuated virus. In this context, the term “reduce/reduced” means a reduction of at least 10%, preferably 25%, even more preferably 50%, still more preferably 60%, even more preferably 70%, still more preferably 80%, still more preferably 90%, even more preferably 95% and most preferably of 100% as compared to the control group infected with non-attenuated IBV as defined above. Thus, an attenuated, IBV strain is one that is suitable for incorporation into an immunogenic composition comprising a modified live IBV.

In another specific aspect of the IBV, H52 IBV or the immunogenic composition according to the present invention the IBV is inactivated.

Any conventional inactivation method can be used for purposes of the present invention. Thus, inactivation can be performed by chemical and/or physical treatments which are known to the person skilled in the art. Preferred inactivation methods include the addition of cyclized binary ethylenimine (BEI) including the addition of a solution of 2-bromoethyleneamine hydrobromide (BEA), which has been cyclized to binary ethylenimine (BEI). Preferred further chemical inactivation agents comprise but are not limited to Triton X-100, Sodium deoxycholate, Cetyltrimethylammonium bromide, β-Propiolactone, Thimerosal, Phenol and Formaldehyde (Formalin). However, the inactivation may also comprise a neutralization step. Preferred neutralization agents include but are not limited to sodium thiosulfate, sodium bisulfite and the alike.

Preferred formalin inactivation conditions include formalin concentration between from about 0.02% (v/v)-2,0% (v/v), more preferably from about 0.1% (v/v)-1,0% (v/v), still more preferably from about 0.15% (v/v)-0.8% (v/v), even more preferably from about 0.16% (v/v)-0.6% (v/v), and most preferably about 0.2% (v/v)-0.4% (v/v). Incubation time depends on the resistance of the IBV. In general, the inaction process is performed until no growth of the IBV can be detected in a suitable cultivation system.

Preferably, the inactivated IBV of the present invention is formalin inactivated, preferably using the concentrations as described hereinabove.

The inactivated IBV of the invention may be incorporated into liposomes using known technology such as that described in Nature, 1974, 252, 252-254 or Journal of Immunology, 1978, 120, 1109-13. In another embodiment of the invention, the inactivated IBV of the invention may be conjugated to suitable biological compounds such as polysaccharides, peptides, proteins, or the like, or a combination thereof.

In another specific aspect of the IBV, H52 IBV or the immunogenic composition according to the present invention the IBV is genetically engineered.

The term “genetically engineered” refers to an IBV which has been mutated by using “reverse genetics” approaches. Preferably, the IBV according to the present invention has been genetically engineered. The reverse genetics technique involves the preparation of synthetic recombinant viral RNAs. However, “reverse genetics” techniques are well known to the person skilled in the art.

In another specific aspect of the IBV, H52 IBV or the immunogenic composition according to the present invention the IBV is a recombinant IBV.

The term “recombinant” as used herein relates to a RNA genome (or RNA sequence, cDNA sequence or protein) having any modifications that do not naturally occur to the corresponding RNA genome (or RNA sequence, cDNA sequence or protein). For instance, a RNA genome (or RNA sequence, cDNA sequence or protein) is considered “recombinant” if it contains an insertion, deletion, inversion, relocation or a point mutation introduced artificially, e.g., by human intervention. Therefore, the RNA genomic sequence (or RNA sequence, cDNA sequence or protein) is not associated with all or a portion of the sequences (or RNA sequence, cDNA sequence or protein) with which it is associated in nature. The term “recombinant” as used with respect to a virus, means a virus produced by artificial manipulation of the viral genome. The term “recombinant virus” encompasses genetically modified viruses.

In another specific aspect of the IBV, H52 IBV or the immunogenic composition according to the present invention the IBV is chimeric.

The term “chimeric” refers to an IBV comprising one or more nucleotide sequences from another coronavirus, preferably from another IBV strain. Exemplary, an IBV H52 encoding for a heterologous S (spike) protein or fragment thereof is a chimeric IBV.

In another specific aspect of the immunogenic composition according to the present invention the immunogenic composition is a vaccine. The term “vaccine” already has been described elsewhere herein. However, in case where the host displays a protective immunological response such that resistance to new infection will be enhanced and/or the clinical severity of the disease reduced, the immunogenic composition is described as a “vaccine.

In another specific aspect of the immunogenic composition according to the present invention the immunogenic composition comprises a pharmaceutically acceptable carrier.

The term “pharmaceutical-acceptable carrier” includes any and all solvents, dispersion media, coatings, stabilizing agents, diluents, preservatives, antibacterial and antifungal agents, isotonic agents, adsorption delaying agents, adjuvants, immune stimulants, and combinations thereof.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

In another specific aspect of the immunogenic composition according to the present invention the pharmaceutically acceptable carrier is phosphate buffered saline.

In some aspects, the immunogenic composition of the present invention contains an adjuvant. “Adjuvants” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.), JohnWiley and Sons, NY, pp51-94 (1995) and Todd et al., Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product.

In another specific aspect of the immunogenic composition according to the present invention the immunogenic composition comprises distilled water.

In another specific aspect of the immunogenic composition according to the present invention the immunogenic composition is effective in the treatment and/or prophylaxis of clinical signs caused by IBV in a subject. The terms “treatment and/or prophylaxis” and “clinical signs” have been defined elsewhere.

In another specific aspect of the immunogenic composition according to the present invention the immunogenic composition protects against a challenge or infection with a DMV/1639 IBV strain.

In another specific aspect of the immunogenic composition according to the present invention said immunogenic composition is formulated for a single-dose administration.

The volume for a single-dose has been defined elsewhere herein.

It has furthermore been shown that one dose of the immunogenic composition of the present invention is effective after the administration of such single dose of such immunogenic composition.

In another specific aspect of the immunogenic composition according to the present invention the immunogenic composition is administered subcutaneously, intramuscularly, oral, in ovo, via spray, via coarse spray, via drinking water, via gel droplet or by eye drop.

In another specific aspect of the immunogenic composition according to the present invention the immunogenic composition is administered via coarse spray or by eye drop.

In another specific aspect of the immunogenic composition according to the present invention the immunogenic composition comprises 1 to 10 log₁₀ EID50 per dose of the IBV.

In another specific aspect of the immunogenic composition according to the present invention the immunogenic composition comprises 2 to 5 log₁₀ EID50 per dose of the IBV.

In another specific aspect of the immunogenic composition according to the present invention the immunogenic composition comprises 2 to 4 log₁₀ EID50 per dose of the IBV.

### Kits

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration preferably for administration to subjects, especially poultry. Associated with such container(s) can be a notice in the form prescribed by a governmental agency regulating the manufacture, use or sale of pharmaceuticals or biological products, which notice reflects approval by the agency of manufacture, use or sale for human administration.

Thus, the present invention provides a kit comprising the IBV or the immunogenic composition as described herein.

In one specific aspect of the kit according to the present invention the kit further comprises an instruction letter for the treatment and/or prophylaxis of diseases of avians.

In one specific aspect of the kit according to the present invention the kit further comprises an instruction letter for the treatment and/or prophylaxis of diseases of poultry.

In one specific aspect of the kit according to the present invention the kit further comprises an instruction letter for the treatment and/or prophylaxis of IB (infectious bronchitis).

In one specific aspect of the kit according to the present invention the kit further comprises an instruction letter for the treatment and/or prophylaxis of DMV/1639 IB.

### Method of Treatments

Further, the present invention provides a method for immunizing a subject comprising administering to such subject an immunogenic composition as described herein.

The term “immunizing” relates to an active immunization by the administration of an immunogenic composition to a subject to be immunized, thereby causing an immunological response against the antigen included in such immunogenic composition.

Preferably, immunization results in lessening of the incidence of the particular IBV infection in a flock or in the reduction in the severity of clinical signs caused by or associated with the particular IBV infection.

Further, the immunization of a subject with the immunogenic compositions as provided herewith, results in preventing infection of a subject by IBV infection. Even more preferably, immunization results in an effective, long-lasting, immunological-response against IBV infection. It will be understood that the said period of time will last more than 1 month, preferably more than 2 months, preferably more than 3 months, more preferably more than 4 months, more preferably more than 5 months, more preferably more than 6 months. It is to be understood that immunization may not be effective in all subjects immunized. However, the term requires that a significant portion of subjects of a flock are effectively immunized.

Preferably, a flock of subjects is envisaged in this context which normally, i.e. without immunization, would develop clinical signs normally caused by or associated with a IBV infection. Whether the subjects of a flock are effectively immunized can be determined without further ado by the person skilled in the art. Preferably, the immunization shall be effective if clinical signs in at least 33%, at least 50%, at least 60%, at least 70%, at least 80%, at least 90%, still more preferably in at least 95% and most preferably in 100% of the subjects of a given flock are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, still more preferably by at least 95% and most preferably by 100% in comparison to subjects that are either not immunized or immunized with an immunogenic composition that was available prior to the present invention but subsequently infected by the particular IBV.

Further, the present invention provides a method of treating or preventing clinical signs caused by IBV in a subject, the method comprising administering to the subject a therapeutically effective amount of an immunogenic composition as described herein.

Further, the present invention provides a method for preventing clinical signs caused by IBV in a subject, the method comprising administering to the subject a therapeutically effective amount of an immunogenic composition as described herein.

The term “treating or preventing” refers to the lessening of the incidence of the particular IBV infection in a flock or the reduction in the severity of clinical signs caused by or associated with the particular IBV infection. Thus, the term “treating or preventing” also refers to the reduction of the number of subjects in a flock that become infected with the particular IBV (=lessening of the incidence of the particular IBV infection) or to the reduction of the severity of clinical signs normally associated with or caused by a IBV infection or the reduction of virus shedding after infection with the particular IBV or preventing or lessening egg drop in laying hens after infection with the particular IBV in a group of subjects which subjects have received an effective amount of the immunogenic composition as provided herein in comparison to a group of subjects which subjects have not received such immunogenic composition.

The “treating or preventing” generally involves the administration of an effective amount of the immunogenic composition of the present invention to a subject or flock of subjects that could benefit from such a treatment/prophylaxis. The term “treatment” refers to the administration of the effective amount of the immunogenic composition once the subject or at least some subjects of the flock is/are already infected with such IBV and wherein such subjects already show some clinical signs caused by or associated with such IBV infection. The term “prophylaxis” refers to the administration of a subject prior to any infection of such subject with IBV or at least where such subject or none of the subjects in a group of subjects do not show any clinical signs caused by or associated with the infection by such IBV. The terms “prophylaxis” and “preventing” are used interchangeable in this application.

The term “an effective amount” as used herein means, but is not limited to an amount of antigen, that elicits or is able to elicit an immune response in a subject. Such effective amount is able to lessen the incidence of the particular IBV infection in a flock or to reduce the severity of clinical signs of the particular IBV infection.

Preferably, clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, still more preferably by at least 95% and most preferably by 100% in comparison to subjects that are either not treated or treated with an immunogenic composition that was available prior to the present invention but subsequently infected by the particular IBV.

The term “clinical signs” as used herein refers to signs of infection of a subject from IBV. Examples for such clinical signs include but are not limited to respiratory distress, nephritis, salphingitis, abnormal egg production, ruffled feathers, depression, reduced growth rates and reduced appetite. Signs of respiratory distress encompass respiratory signs including gasping, coughing, sneezing, tracheal rales, nasal and ocular discharge, tracheal lesions and ciliostasis in the trachea. Signs of nephritis encompass kidney lesions and watery diarrhea. Signs of abnormal egg production encompass egg drop, eggs of smaller size, inferior shell, reduced internal egg quality, eggs with thin albumen and ciliostasis in the oviduct. However, the clinical signs also include but are not limited to clinical signs that are directly observable from a live animal. Examples for clinical signs that are directly observable from a live animal include nasal and ocular discharge, coughing, gasping, sneezing, tracheal rales, ruffled feathers, conjunctivitis, weight loss, reduced growth rates, reduced appetite, dehydration, watery diarrhea, lameness, lethargy, wasting and unthriftiness and the like.

Preferably, the clinical signs lessened in incidence or severity in a treated subject compared to subjects that are either not treated or treated with an immunogenic composition that was available prior to the present invention but subsequently infected by the particular IBV refer to a reduction of ciliostasis, a reduction of rales, a reduction of egg drop, a reduction of kidney lesions, a reduction of watery diarrhea, a reduction in weight loss, a lower virus load, a reduced viral shedding, or combinations thereof.

The term “reducing” or or “reduced” or “reduction” or lower” are used interchangeable in this application. The term “reduction” means, that the clinical sign is reduced by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, even more preferably by at least 90%, still more preferably by at least 95% most preferably by 100% in comparison to subjects that are not treated (not immunized) but subsequently infected by the particular IBV.

Further, the present invention provides a method of reducing the ciliostasis in a subject, in comparison to a subject of a non-immunized control group of the same species, the method comprising administering to the subject a therapeutically effective amount of an immunogenic composition as described herein.

The term “ciliostasis” refers to a reduced movement of the cilia in the trachea. Thus, ciliostasis may be determined by examining the inner lining of the tracheal rings for the movement of the cilia. It is in the general knowledge of a person skilled in the art how to determine the movement of the cilia in the trachea.

Preferably, the movement of the cilia is not reduced from day 10 after challenge or infection, more preferably from day 5 after challenge or infection, more preferably from day 4 after challenge or infection, more preferably from day 3 after challenge or infection and most preferably from day 1 or 2 after challenge or infection with the IBV as compared to a subject of a non-immunized control group of the same species.

The term “reduction of ciliostasis” means, that the ciliostasis is reduced by at least 10%, preferably by at least 20%, more preferably by at least 30%, even more preferably by at least 40%, even more preferably by at least 50%, even more preferably by at least 60%, even more preferably by at least 70%, even more preferably by at least 80%, even more preferably by at least 90%, even more preferably by at least 95% and most preferably by 100% as compared to a subject of a non-immunized control group of the same species. It is in the general knowledge of a person skilled in the art how to measure the reduction of the ciliostasis.

Further, the present invention provides a method of reducing viral load in a subject, in comparison to a subject of a non-immunized control group of the same species, the method comprising administering to the subject a therapeutically effective amount of an immunogenic composition as described herein.

As shown in the Examples, the immunogenic composition as provided herein has been proven to be efficacious in reducing the viral load.

Further, the present invention provides the immunogenic composition as described herein for use in a method for immunizing a subject, the method comprising administering to the subject a therapeutically effective amount of said immunogenic composition

Further, the present invention provides the immunogenic composition as described herein for use in a method of treating or preventing clinical signs caused by IBV in a subject, the method comprising administering to the subject a therapeutically effective amount of said immunogenic composition.

Further, the present invention provides the immunogenic composition as described herein for use in a method of reducing the ciliostasis in a subject, in comparison to a subject of a non-immunized control group of the same species, the method comprising administering to the subject a therapeutically effective amount of said immunogenic composition.

Further, the present invention provides the immunogenic composition as described herein for use in method of reducing viral load in a subject, in comparison to a subject of a non-immunized control group of the same species, the method comprising administering to the subject a therapeutically effective amount of said immunogenic composition.

In one specific aspect of the method or use according to the present the present invention said subject is avian.

The term “avian” is well known to the person skilled in the art. The term “avian” encompasses all birds including poultry.

In another specific aspect of the method or use according to the present invention said subject is poultry.

The term “poultry” is well known to the person skilled in the art. The term “poultry” encompasses chickens, turkeys, quails, pheasants, guineafowl, geese, and ducks. Further, the term “chicken” includes broiler, laying hens, and reproductive stocks for both also refeered as breeders.

In another specific aspect of the method or use according to the present invention said subject is selected from the list consisting of chicken, turkey, quail, or pheasant.

In another specific aspect of the method or use according to the present invention said subject is chicken.

In another specific aspect of the method or use according to the present invention the immunogenic composition is administered once.

It is understood, that a single-dose is administered only once. As shown in the Examples the immunogenic composition as provided herein has been proven to be efficacious after the administration of a single dose to a subject of need.

The dose volume per poultry depends on the route of vaccination and the age of the poultry.

Typically, eye drop vaccines are administered in a volume of 1 to 100 μl per dose at any age. Preferably, the single-dose for eye drop vaccines has a total volume between about 5 μl and 70 μl and more preferably between about 20 μl and 50 μl with a single 20 μl, 25 μl, 30 μl, 35 μl, 40 μl, 45 μl or 50 μl dose being preferred. Most preferred, the single-dose for eye drop vaccines has a total volume between between about 30 μl and 50 μl with a single 30 μl, 35 μl, 40 μl, 45 μl or 50 μl dose being preferred.

Spray vaccines may contain the dose in a volume of 25 to 1000 μl for day-old poultry. Preferably, the single-dose for spray vaccines has a total volume between about 50 μl and 5000 μl, more preferably between about 75 μl and 2000 μl, more preferably between about 100 μl and 1000 μl, even more preferably between about 200 μl and 900 μl, even more preferably between about 300 μl and 800 μl and even more preferably between about 400 μl and 700 μl with a single 400 μl, 425 μl, 450 μl, 475 μl, 500 μl, 525 μl, 550 μl, 575 μl, 600 μl, 625 μl, 650 μl, 675 μl or 700 μl dose being preferred. Most preferred the single-dose has a total volume of 400 μl, 450 μl 500 μl, 550 μl, 600 μl, 650 μl or 700 μl.

The vaccine for intramuscular or subcutaneous vaccination or one dose of a drinking water vaccine may contain the dose in a volume of 30 μl to 1000 μl. Preferably, the single-dose has a total volume between about 30 μl and 1000 μl, more preferably between about 50 μl and 500 μl, more preferably between about 75 μl and 250 μl and even more preferably between about 100 μl and 200 μl with a single 100 μl, 110 μl, 120 μl, 125 μl, 130 μl, 135 μl, 140 μl, 145 μl, 150 μl, 160 μl, 170 μl, 175 μl, 180 μl, 190 μl, 155 μl, or 200 μl dose being the most preferred.

In another specific aspect of the method or use according to the present invention the immunogenic composition is administered at two or more doses.

However, the immunogenic composition can be administered at two or more doses, with a first dose being administered prior to the administration of a second (booster) dose.

In a preferred aspect of the two-time administration regimen, both the first and second doses of the immunogenic composition are administered in the same amount. Preferably, each dose is in the preferred amounts specified above. In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned above.

Preferably, the first administration of the vaccine is performed within the first three weeks of age, more preferably within the first week of age and most preferred at one day-of-age by methods as described below. A second administration can be performed within the first 20 weeks of age, preferably within 16-18 weeks of age, more preferably between 6-12 weeks of age. Exemplary, the initial (first) vaccination is performed at 1-10 days of age and the second vaccination (booster) is performed at 6-12 or 16-18 weeks of age. More preferably, the initial (first) vaccination is performed at one day-of-age and the second vaccination (booster) is performed at 6-12 or 16-18 weeks of age.

In another specific aspect of the method or use according to the present invention said immunogenic composition is administered subcutaneously, intramuscularly, oral, in ovo, via spray, via coarse spray, via drinking water, via gel droplet or by eye drop.

The immunogenic composition is, preferably, administered topically or systemically. Suitable routes of administration conventionally used are oral or parenteral administration, such as intranasal, intravenous, intradermal, transdermal, intramuscular, intraperitoneal, subcutaneous, as well as inhalation, in ovo, via spray, via drinking water or by eye drop. The preferred spray method is coarse spray. However, depending on the nature and mode of action of a compound, the immunogenic composition may be administered by other routes as well. For example, such other routes include intracutaneously, intravenously, intravascularly, intraarterially, intraperitnoeally, intrathecally, intratracheally, intracutaneously, intracardially, intralobally, intralobarly, intramedullarly, intrapulmonarily, intrarectally, and intravaginally. However, most preferred the immunogenic composition is administered subcutaneously, intramuscularly, oral, in ovo, via spray, via coarse spray, via drinking water, via gel droplet or by eye drop.

Live IBV vaccines are preferably administered individually by eye drop, intranasal, intramuscular or subcutaneous.

More preferably, mass application methods, including drinking water and aerosol spray vaccination, are used. Preferably, coarse spray is used.

In another specific aspect of the method or use according to the present invention said immunogenic composition is administered via spray.

In another specific aspect of the method or use according to the present invention said immunogenic composition is administered via coarse spray.

For example, broilers may be vaccinated at one-day of age or at 1-3 weeks of age, particularly for broilers with high levels of MDA. Laying stock or reproduction stock may be vaccinated initially at 1-10 days of age and boosted with the vaccine at 7-12 or 16-18 weeks of age.

In another specific aspect of the method or use according to the present invention said immunogenic composition is administered via eye drop.

Typically, the live vaccine for post-hatch administration comprises the attenuated IBV in a concentration of 10¹ to 10⁸ EID₅₀ (50% Egg Infective Dose) per dose, preferably in a concentration of 10² to 10⁵ EID₅₀ per dose and, more preferably, in a concentration of 10² to 10⁴ EID₅₀ per dose and, even more preferably, in a concentration of 10² to 10³ EID₅₀ per dose.

Preferably, the immunogenic composition of the present invention comprises the IBV of the present invention in amounts of about 1 to about 10 log₁₀ EID (egg infective dose)₅₀ per dose, preferably about 2 to about 8 log₁₀ EID₅₀ per dose, preferably in an amount of about 2 to about 7 log₁₀ EID₅₀ per dose, more preferably in an amount of about 2 to about 6 log₁₀ EID₅₀ per dose, even more preferably in an amount of about 2 to about 5 log₁₀ EID₅₀ per dose, even more preferably in an amount of about 2 to about 4 log₁₀ EID₅₀ per dose, most preferably in an amount of about 2 to about 3 log₁₀ EID₅₀ per dose. More preferably, the immunogenic composition of the present invention comprises the IBV of the present invention in amounts of about 1, 1.5, 2, 2.5, 3, 3.5, 4, 4.5, 5, 5.5, 6, 6.5, 7, 7.5 or log₁₀ EID₅₀ per dose.

In another specific aspect of the method or use according to the present invention the immunogenic composition comprises 1 to 10 log₁₀ EID₅₀ per dose of the IBV.

In another specific aspect of the method or use according to the present invention the immunogenic composition comprises 2 to 5 log₁₀ EID₅₀ per dose of the IBV.

In another specific aspect of the method or use according to the present invention the immunogenic composition comprises 2 to 4 log₁₀ EID₅₀ per dose of the IBV.

In another specific aspect of the method or use according to the present invention the immunogenic composition is administered to subjects within the first week of age, within the first three days of age, within the first two days of age, or within the first day of age.

Preferably, the subject to be immunized is 1, 2, 3, 4, 5, 6, 7, 8, 9, 10, 11, 12, 13, 14, 15, 16, 17, 18, 19, 20 or 21 days of age. More preferably, said subject to be immunized is 1, 2, 3, 4, 5, 6, 7, 8, 9, 10, 11, 12, 13 or 14 days of age. Most preferably, said subject to be immunized is 1, 2, 3, 4, 5, 6 or 7 days of age.

However, it has to be understood that after vaccination of the subject being a few days of age, it does need several days for the immune system of the poultry to build up immunity against an IBV infection. Therefore, preferably, the subjects are immunized within the first 24 h of age.

In another specific aspect of the method or use according to the present invention the immunogenic composition is administered to subjects within the first day of age. As shown in the Examples the immunogenic composition as provided herein has been proven to be safe and efficacious when administered to 1-day old poultry.

In another specific aspect of the method or use according to the present invention said method results in an improvement in an efficacy parameter selected from the group consisting of: prevention or reduction of ciliostasis, prevention or reduction of rales, prevention or reduction of egg drop, prevention or reduction of kidney lesions, prevention or reduction of watery diarrhea, prevention or reduction in weight loss, a lower virus load, a reduced viral shedding or combinations thereof, in comparison to a subject of a non-treated control group of the same species.

The terms “treatment and/or prophylaxis” have been defined elsewhere, wherein the terms “prophylaxis” and “preventing” or “prevention” are used interchangeable in this application. Further, the terms “shedding” has been defined elsewhere, too.

The term “reducing”, “reduced”, “reduction” or “lower” means, that the efficacy parameter (ciliostasis, rales, egg drop, kidney lesions, watery diarrhea, weight loss, virus load, viral shedding) is reduced by at least 10%, preferably by at least 20%, more preferably by at least 30%, even more preferably by at least 40%, even more preferably by at least 50%, even more preferably by at least 60%, even more preferably by at least 70%, even more preferably by at least 80%, even more preferably by at least 90%, even more preferably by at least 95% and most preferably by 100% as compared to a subject of a non-immunized control group of the same species. It is in the general knowledge of a person skilled in the art how to measure the improvement in the efficacy parameters.

The term “virus load” is well known to the person skilled in that art. The term virus load is interchangeable used with the term viral titer herein. The virus load or virus titer is a measure of the severity of an active viral infection, and can be determined by methods known to the person skilled in the art. The determination can be based on the detection of viral proteins such as by antibody binding to the viral proteins and further detection or, alternatively, by detection of viral RNA by amplification methods such as RT-PCR. Monitoring of virion associated viral RNA in plasma by nucleic acid amplification methods is a widely used parameter to assess the status and progression of retroviral disease, and to evaluate the effectiveness of prophylactic and therapeutic interventions. Exemplary, the virus load or virus titer can be calculated by estimating the live amount of virus in an involved body fluid such as a number of RNA copies per milliliter of blood plasma.

The term “ciliostasis” is well known to the person skilled in that art. The surface of the trachea is covered with specialised epithelial cells, which are lined with numerous, motile, hair-like structures called cilia. The term “ciliostasis” encompasses the reduction or loss of cilia and/or loss or partial loss of ciliary activity. Ciliostasis can be determined without further ado by the person skilled in the art.

The term “rales” is well known to the person skilled in that art. However, the term “rales” encompasses tracheal rales and refers to sounds emanating from the bronchi. Rales can be determined without further ado by the person skilled in the art.

The term “egg drop” is well known to the person skilled in that art. The term “egg drop” encompasses a decreased egg production.

In another specific aspect of the method or use according to the present invention the treatment or prevention results in a prevention or reduction of ciliostasis as compared to subjects of a non-treated control group of the same species.

In another specific aspect of the method or use according to the present invention the treatment or prevention results in a prevention or reduction of kidney lesions as compared to subjects of a non-treated control group of the same species.

In another specific aspect of the method or use according to the present invention the treatment or prevention results in a prevention or reduction of egg drop as compared to subjects of a non-treated control group of the same species.

In another specific aspect of the method or use according to the present invention the treatment or prevention results in a prevention or reduction of the viral load in a subject as compared to subjects of a non-treated control group of the same species.

The present invention further provides an IBV or an immunogenic composition as described herein for therapeutic use.

The present invention further provides an IBV or an immunogenic composition as described herein for use as an immunogen or vaccine.

The present invention further provides an IBV or an immunogenic composition as described herein for use as a medicament.

The present invention further provides the use of the IBV or immunogenic composition as described herein for the manufacture of a medicament.

The present invention further provides the use of the IBV or immunogenic composition as described herein for the treatment and/or prophylaxis of IBV infections in a subject.

The present invention further provides an immunogenic composition comprising an H52 IBV (infectious bronchitis virus) encoding for a heterologous DMV/1639 S (spike) protein, wherein said H52 IBV comprises a Nucleocapsid (N) protein, Envelope (E) protein or Membrane glycoprotein (M) having or consisting of or comprising the amino acid sequence as shown for AAK91809 (SEQ ID NO 6), AAK27163 (SEQ ID NO:7), SEQ ID NO 8, AAL26888 (SEQ ID NO:9), SEQ ID NO 10, AAK83031 (SEQ ID NO:11) or SEQ ID NO 12 or a sequence having at least 90%, 95%, 96%, 97%, 98%, 99%, 99.2%, 99.5%, 99.6%, 99.7%, 99.8%, 99.9%, 99.95%, 99.98% or 99.99% sequence identity thereto, and, wherein the heterologous DMV/1639 S protein is consisting of or comprising the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.

In another specific aspect of the immunogenic composition according to the present invention the heterologous S protein is the full length spike protein.

In another specific aspect of the immunogenic composition according to the present invention the fragment of the heterologous S (spike) protein has a length of at least 1000 or 1077 amino acids from the N-Terminus.

In another specific aspect of the immunogenic composition according to the present invention the fragment of the heterologous S (spike) protein is the Ectodomain of the spike protein.

In another specific aspect of the immunogenic composition according to the present invention the IBV is attenuated.

The present invention further provides a method of preparing an immunogenic composition for the treatment and/or prophylaxis of IBV infections in a subject comprising:
a.) providing an H52 IBV comprising a spike (S) protein, nucleocapsid (N) protein, envelope (E) protein or membrane glycoprotein (M) having the amino acid sequence as shown for for AAK27168 (SEQ ID NO:4), SEQ ID NO:5, AAK91809 (SEQ ID NO:6), AAK27163 (SEQ ID NO:7), SEQ ID NO:8, AAL26888 (SEQ ID NO:9), SEQ ID NO:10, AAK83031 (SEQ ID NO:11) or SEQ ID NO:12 or a sequence having at least 90%, 95%, 96%, 97%, 98%, 99%, 99.2%, 99.5%, 99.6%, 99.7%, 99.8%, 99.9%, 99.95%, 99.98% or 99.99% sequence identity thereto; and
b.) providing a heterologous DMV/1639 S protein having or consisting of or comprising the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof; and
c.) replacing the spike protein of H52 IBV of a) with said heterologous DMV/1639 S (spike) protein of b) to have an H52 IBV with a heterologous DMV/1639 S protein; and
d.) obtaining said H52 IBV with a heterologous S protein; and
e.) addition of a pharmaceutically acceptable carrier.

The term “obtaining” comprises the harvest, isolation, purification and/or formulation (e.g. finishing, inactivation and/or blending) of said H52 IBV with a heterologous DMV/1639 S protein or fragment thereof.

The term “harvest” refers to collecting or recovering said said H52 IBV with a heterologous DMV/1639 S protein or fragment thereof from the transfected or infected cell or cell line. Any conventional method known in the art can be used, e.g. any separation method. Well known methods in the art comprise centrifugation or filtration, such as using a semi-permeable membrane having a certain pore size.

The term “isolation” comprises an isolation step of said H52 IBV with a heterologous DMV/1639 S protein or fragment thereof. Methods for the isolation from the transfected or infected cell or cell line are known to a person skilled in the art. Those methods comprise physical and/or chemical methods, including but are not limited to freeze thaw cycles, treatment with ultrasound and the alike.

Methods for the “purification” of said said H52 IBV with a heterologous DMV/1639 S protein or fragment thereof from the isolate are known to a person skilled in the art, for example by those methods described in Protein purification methods—a practical approach (E. L. V. Harris and S. Angel, eds., IRL Press at Oxford University Press). Those methods include, but are not limited to, separation by centrifugation and/or filtration, precipitation, size exclusion (gel filtration) chromatography, affinity chromatography, metal chelate chromatography, ion-exchange chromatography covalent chromatography, hydrophobic interaction chromatography, and the alike. The vector can be obtained in a purified pure form, or free or substantially free of other cellular materials or culture medium etc. After said isolation and/or purification the antigen exhibits a purity of at least 80%, preferably 80%-90%, more preferably 90%-97%, most preferred more than 97% up to an absolute pure form without any contamination.

According to a further aspect, “obtaining” as used herein may also include further finishing steps as part of the final formulation process, like the addition of buffer, inactivation, neutralization steps and the alike.

In another specific aspect of the method of preparing an immunogenic composition according to the present invention, the heterologous DMV/1639 S (spike) protein is the full length Spike protein.

In another specific aspect of the method of preparing an immunogenic composition according to the present invention, the heterologous DMV/1639 S (spike) protein is the Ectodomain of the Spike protein.

In another specific aspect of the method of preparing an immunogenic composition according to the present invention, said pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.

In another specific aspect of the method of preparing an immunogenic composition according to the present invention, the IBV is attenuated.

The present invention further concerns a plasmid comprising a nucleic acid encoding a partial H52 IBV (infectious bronchitis virus) genome including a heterologous IBV S (spike) protein or fragment thereof, such as the pUC57-Brick H52 rIBV DMV S donor plasmid (SEQ ID NO:14).

The present invention further provides the IBV, H52 IBV or immunogenic composition as described herein for use as a coarse spray vaccine or for use in coarse spray application.

### CLAUSES

The following clauses are also described herein:
1. An IBV (infectious bronchitis virus) encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.
2. A H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.
3. A H52 IBV (infectious bronchitis virus) encoding for a heterologous DMV/1639 IBV spike protein or fragment thereof.
4. An immunogenic composition comprising a H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.
5. An immunogenic composition comprising an IBV (infectious bronchitis virus) of clauses 1 to 3.
6. The IBV or the immunogenic composition of clauses 1 and 5, wherein the IBV is selected from the group consisting of: H52, H120, 4/91 and QX.
7. The IBV or the immunogenic composition of any one of clauses 1, 5 and 6, wherein the IBV is H52 or H120.
8. The IBV, H52 IBV or the immunogenic composition of any one of clauses 2 to 7, wherein the H52 IBV has or consists of or comprises a nucleotide sequence as shown for EU817497 (SEQ ID NO:3) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
9. The IBV, H52 IBV or the immunogenic composition of any one of clauses 2 to 8, wherein the H52 IBV strain has or consists of or comprises a spike (S1) protein having the amino acid sequence as shown for AAK27168 (SEQ ID NO:4) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
10. The IBV, H52 IBV or the immunogenic composition of any one of clauses 2 to 9, wherein the H52 IBV strain has or consists of or comprises a spike (S) protein having the amino acid sequence as shown SEQ ID NO:5 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
11. The IBV, H52 IBV or the immunogenic composition of any one of clauses 2 to 10, wherein the H52 IBV has or consists of or comprises a nucleocapsid (N) protein having the amino acid sequence as shown for AAK91809 (SEQ ID NO:6) or AAK27163 (SEQ ID NO:7) a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
12. The IBV, H52 IBV or the immunogenic composition of any one of clauses 2 to 11, wherein the H52 IBV has or consists of or comprises a nucleocapsid (N) protein having the amino acid sequence as shown SEQ ID NO:8 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
13. The IBV, H52 IBV or the immunogenic composition of any one of clauses 2 to 12, wherein the H52 IBV has or consists of or comprises an envelope (E) protein having the amino acid sequence as shown for AAL26888 (SEQ ID NO:9) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
14. The IBV, H52 IBV or the immunogenic composition of any one of clauses 2 to 13, wherein the H52 IBV has or consists of or comprises an envelope (E) protein having the amino acid sequence as shown in SEQ ID NO:10 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
15. The IBV, H52 IBV or the immunogenic composition of any one of clauses 2 to 14, wherein the H52 IBV has or consists of or comprises a membrane glycoprotein (M) protein having the amino acid sequence as shown for AAK83031 (SEQ ID NO:11) or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
16. The IBV, H52 IBV or the immunogenic composition of any one of clauses 2 to 15, wherein the H52 IBV has a or consists of or comprises membrane glycoprotein (M) protein having the amino acid sequence as shown in SEQ ID NO:12 or a sequence having at least 95%, 96%, 97%, 98%, 99%, 99.2%, 99.5%, 99.6%, 99.7%, 99.8%, 99.9%, 99.95%, 99.98% or 99.99% sequence identity thereto.
17. The IBV, H52 IBV or the immunogenic composition of any one of clauses 1 to 16, wherein the DMV/1639 spike consists of or comprises a sequence selected from the list consisting of: SEQ ID NO: 20 (QCX19619), SEQ ID NO: 22 (QGM12378), SEQ ID NO: 23 (QGM12391), SEQ ID NO: 24 (QGM12404), SEQ ID NO: 25 (QGM12416), SEQ ID NO: 26 (QGM12429), SEQ ID NO: 27 (QRG28806), SEQ ID NO: 28 (QRG28819), SEQ ID NO: 29 (UXN85484), SEQ ID NO: 30 (UXN85494), SEQ ID NO: 31 (UXN85504) and SEQ ID NO: 32 (UZP65106) or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto.
18. The IBV, H52 IBV or the immunogenic composition of any one of clauses 1 to 17, wherein the DMV/1639 spike consists of or comprises the sequence as shown in SEQ ID NO: 20 (QCX19619) or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.
19. The IBV, H52 IBV or the immunogenic composition of any one of clauses 1 to 18, wherein the heterologous DMV/1639 S protein has at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 1, 2, 20 or 21 or the heterologous DMV/1639 S protein consists of or comprises the amino acid sequence as shown in SEQ ID NO: 1, 2, 20 or 21 or a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.
20. The IBV, H52 IBV or the immunogenic composition of any one of clauses 1 to 19, wherein the heterologous DMV/1639 S protein is selected from a list consisting of SEQ ID NO: 1, 2, 20 or 21.
21. The IBV, H52 IBV or the immunogenic composition of any one of clauses 1 to 20, wherein the heterologous DMV/1639 S protein has one or more amino acid(s) selected from the list consisting of: 242T, 489F and 1038L.
22. The IBV, H52 IBV or the immunogenic composition of any one of clauses 1 to 21, wherein the heterologous DMV/1639 S protein has the amino acid 242T and/or 489F and/or 1038L.
23. The IBV, H52 IBV or the immunogenic composition of any one of clauses 1 to 22, wherein the heterologous DMV/1639 S protein has the amino acid 242T and 489F and 1038L.
24. The IBV, H52 IBV or the immunogenic composition of any one of clauses 1 to 23, wherein the numbering of the amino acid positions refer to the amino acid positions in a DMV/1639 spike protein.
25. The IBV, H52 IBV or the immunogenic composition of any one of clauses 1 to 24, wherein the numbering of the amino acid positions 242T, 489F and 1038L refer to the amino acid positions in a DMV/1639 spike protein.
26. The IBV, H52 IBV or the immunogenic composition of any one of clauses 1 to 25, wherein the heterologous DMV/1639 S protein has the amino acid Cysteine at amino acid position 270 or the heterologous DMV/1639 S protein has the amino acid 270C.
27. The IBV, H52 IBV or the immunogenic composition of any one of clauses 1 to 26, wherein the heterologous DMV/1639 S protein has the amino acid L270C.
28. The IBV, H52 IBV or the immunogenic composition of clauses 26 or 27, wherein the Cysteine at amino acid position 270 is introduced by a mutation.
29. The IBV, H52 IBV or the immunogenic composition of any one of clauses 1 to 28, wherein the Cysteine at amino acid position 270 leads to an extended cell or tissue tropism of the IBV.
30. The IBV, H52 IBV or the immunogenic composition of any one of clauses 1 to 29, wherein the numbering of amino acid positions refer to the amino acid positions in the spike protein as given in SEQ ID NO:1.
31. The IBV, H52 IBV or the immunogenic composition of any one of clauses 1 to 30, wherein the numbering of amino acid positions 242T, 270C, 489F and 1038L refer to the amino acid positions 242T, 270C, 489F and 1038L in the spike protein as given in SEQ ID NO:1.
32. The IBV, H52 IBV or the immunogenic composition of any one of clauses 1 to 31, wherein the amino acid sequence of SEQ ID NO:1 is used for determining the position numbering in the spike protein.
33. The IBV, H52 IBV or the immunogenic composition of any one of clauses 1 to 32, wherein the amino acid sequence of SEQ ID NO:1 is used for determining the position numbering of 242T, 270C, 489F and 1038L in the spike protein.
34. The IBV, H52 IBV or the immunogenic composition of any one of clauses 1 to 33, wherein for determining the amino positions in a spike protein the amino acid sequence is aligned to the amino acid sequence of SEQ ID NO:1.
35. The IBV, H52 IBV or the immunogenic composition of any one of clauses 1 to 34, wherein for determining the amino positions 242T, 270C, 489F and 1038L in a spike protein the amino acid sequence is aligned to the amino acid sequence of SEQ ID NO:1.
36. The IBV, H52 IBV or the immunogenic composition of any one of clauses 1 to 35, wherein the amino acid positions 242T, 270C and 489F are within the S1 subunit of the spike protein and 1038L is within the S2 subunit of the spike protein.
37. The IBV, H52 IBV or the immunogenic composition of any one of clauses 1 to 36, wherein the heterologous S protein is the full length Spike protein.
38. The IBV, H52 IBV or the immunogenic composition of any one of clauses 1 to 37, wherein the fragment of the heterologous DMV/1639 S (spike) protein has a length of at least 1000, at least 1050 or at least 1077 amino acids.
39. The IBV, H52 IBV or the immunogenic composition of any one of clauses 1 to 37, wherein the fragment of the heterologous DMV/1639 S (spike) protein has a length of at least 1000, at least 1050 or 1077 amino acids from the N-Terminus.
40. The IBV, H52 IBV or the immunogenic composition of any one of clauses 1 to 39, wherein the fragment of the heterologous DMV/1639 S (spike) protein has a length of at least 1000 amino acids.
41. The IBV, H52 IBV or the immunogenic composition of any one of clauses 1 to 40, wherein the fragment of the heterologous S (spike) protein is the ectodomain of the spike protein.
42. The IBV, H52 IBV or the immunogenic composition of any one of clauses 1 to 41, wherein the heterologous DMV S (spike) protein replaces the homologous S protein.
43. The IBV, H52 IBV or the immunogenic composition of any one of clauses 1 to 42, wherein the heterologous DMV/1639 S (spike) protein replaces the natural occuring S protein.
44. The IBV, H52 IBV or the immunogenic composition of any one of clauses 1 to 43, wherein the heterologous S (spike) protein replaces the S protein in H52, H120, 4/91 or QX.
45. The IBV, H52 IBV or the immunogenic composition of any one of clauses 1 to 44, wherein the heterologous S (spike) protein replaces the S protein in H52.
46. The IBV, H52 IBV or the immunogenic composition of any one of clauses 1 to 45, wherein the IBV is attenuated.
47. The IBV, H52 IBV or the immunogenic composition of any one of clauses 1 to 46, wherein the IBV is inactivated.
48. The IBV, H52 IBV or the immunogenic composition of any one of clauses 1 to 47, wherein the IBV is genetically engineered.
49. The IBV, H52 IBV or the immunogenic composition of any one of clauses 1 to 48, wherein the IBV is a recombinant IBV.
50. The immunogenic composition of any one of clauses 4 to 49, wherein the immunogenic composition is a vaccine.
51. The immunogenic composition of any one of clauses 4 to 50, wherein the immunogenic composition comprises a pharmaceutically acceptable carrier.
52. The immunogenic composition of clause 51, wherein the pharmaceutically acceptable carrier is phosphate buffered saline.
53. The immunogenic composition of any one of clauses 4 to 52, wherein the immunogenic composition comprises distilled water.
54. The immunogenic composition of any one of clauses 4 to 53, wherein the immunogenic composition is effective in the treatment and/or prophylaxis of clinical signs caused by IBV in a subject.
55. The immunogenic composition of any one of clauses 4 to 54, wherein the immunogenic composition protects against a challenge or infection with a DMV/1639 IBV strain.
56. The immunogenic composition of any one of clauses 4 to 55, wherein said immunogenic composition is formulated for a single-dose administration.
57. The immunogenic composition of any one of clauses 4 to 56, wherein said immunogenic composition is administered subcutaneously, intramuscularly, oral, in ovo, via spray, via coarse spray, via drinking water, via gel droplet or by eye drop.
58. The immunogenic composition of any one of clauses 4 to 57, wherein the immunogenic composition comprises 1 to 10 log₁₀ EID₅₀ per dose of the IBV.
59. The immunogenic composition of any one of clauses 4 to 58, wherein the immunogenic composition comprises 2 to 5 log₁₀ EID₅₀ per dose of the IBV.
60. The immunogenic composition of any one of clauses 4 to 59, wherein the immunogenic composition comprises 2 to 4 log₁₀ EID₅₀ per dose of the IBV.
61. A kit comprising the IBV, H52 IBV or the immunogenic composition of any one of clauses 1 to 60.
62. The kit according to clause 61, wherein the kit further comprises an instruction letter for the treatment and/or prophylaxis of diseases of avians.
63. The kit according to clause 61, wherein the kit further comprises an instruction letter for the treatment and/or prophylaxis of diseases of poultry.
64. The kit according to clauses 61, wherein the kit further comprises an instruction letter for the treatment and/or prophylaxis of IB.
65. The kit according to clauses 61, wherein the kit further comprises an instruction letter for the treatment and/or prophylaxis of DMV/1639 IB.
66. A method for immunizing a subject comprising administering to such subject an immunogenic composition according to any one of clauses 4 to 60.
67. A method of treating or preventing clinical signs caused by IBV in a subject, the method comprising administering to the subject a therapeutically effective amount of an immunogenic composition according to any one of clauses 4 to 60.
68. A method of reducing the ciliostasis in a subject, in comparison to a subject of a non-immunized control group of the same species, the method comprising administering to the subject a therapeutically effective amount of an immunogenic composition according to any one of clauses 4 to 60.
69. A method of reducing viral load in a subject, in comparison to a subject of a non-immunized control group of the same species, the method comprising administering to the subject a therapeutically effective amount of an immunogenic composition according to any one of clauses 4 to 60.
70. The immunogenic composition according to any one of clauses 4 to 60 for use in a method for immunizing a subject, the method comprising administering to the subject a therapeutically effective amount of said immunogenic composition.
71. The immunogenic composition according to any one of clauses 4 to 60 for use in a method of treating or preventing clinical signs caused by IBV in a subject, the method comprising administering to the subject a therapeutically effective amount of said immunogenic composition.
72. The immunogenic composition according to any one of clauses 4 to 60 for use in a method of reducing the ciliostasis in a subject, in comparison to a subject of a non-immunized control group of the same species, the method comprising administering to the subject a therapeutically effective amount of said immunogenic composition.
73. The immunogenic composition according to any one of clauses 4 to 60 for use in method of reducing viral load in a subject, in comparison to a subject of a non-immunized control group of the same species, the method comprising administering to the subject a therapeutically effective amount of said immunogenic composition.
74. The method or use of any one of clauses 66 to 73, wherein said subject is avian.
75. The method or use of any one of clauses 66 to 74, wherein said subject is poultry.
76. The method or use of any one of clauses 66 to 75, wherein said subject is selected from the list consisting of chicken, turkey, quail, or pheasant.
77. The method or use of any one of clauses 66 to 76, wherein said subject is chicken.
78. The method or use of any one of clauses 66 to 77, wherein the immunogenic composition is administered once.
79. The method or use of any one of clauses 66 to 78, wherein the immunogenic composition is administered at two or more doses.
80. The method or use of any one of clauses 66 to 79, wherein said immunogenic composition is administered subcutaneously, intramuscularly, oral, in ovo, via spray, via coarse spray, via drinking water, via gel droplet or by eye drop.
81. The method or use of any one of clauses 66 to 80, wherein said immunogenic composition is administered via eye drop or by coarse spray.
82. The method or use of any one of clauses 66 to 81, wherein the immunogenic composition comprises 1 to 10 log₁₀ EID₅₀ per dose of the IBV.
83. The method or use of any one of clauses 66 to 82, wherein the immunogenic composition comprises 2 to 5 log₁₀ EID₅₀ per dose of the IBV.
84. The method or use of any one of clauses 66 to 83, wherein the immunogenic composition comprises 2 to 4 log₁₀ EID₅₀ per dose of the IBV.
85. The method or use of any one of clauses 66 to 84, wherein the immunogenic composition is administered to subjects within the first week of age, within the first three days of age, within the first two days of age, or within the first day of age.
86. The method or use of any one of clauses 66 to 85, wherein the immunogenic composition is administered to subjects within the first day of age.
87. The method or use of any one of clauses 66 to 86, wherein said method results in an improvement in an efficacy parameter selected from the group consisting of: prevention or reduction of ciliostasis, prevention or reduction of rales, prevention or reduction of egg drop, prevention or reduction of kidney lesions, prevention or reduction of watery diarrhea, prevention or reduction in weight loss, a lower virus load, a reduced viral shedding or combinations thereof, in comparison to a subject of a non-treated control group of the same species.
88. The method or use of any one of clauses 66 to 87, wherein the treatment or prevention results in a prevention or reduction of ciliostasis as compared to subjects of a non-treated control group of the same species.
89. The method or use of any one of clauses 66 to 88, wherein the treatment or prevention results in a prevention or reduction of kidney lesions as compared to subjects of a non-treated control group of the same species.
90. The method or use of any one of clauses 66 to 89, wherein the treatment or prevention results in a prevention or reduction of egg drop as compared to subjects of a non-treated control group of the same species.
91. The method or use of any one of clauses 66 to 90, wherein the treatment or prevention results in a prevention or reduction of the viral load in a subject as compared to subjects of a non-treated control group of the same species.
92. The IBV, H52 IBV or immunogenic composition of any one of clauses 1 to 60 for therapeutic use.
93. The IBV, H52 IBV or immunogenic composition of any one of clauses 1 to 60 for use as an immunogen or vaccine.
94. The IBV, H52 IBV or immunogenic composition of any one of clauses 1 to 60 for use as a medicament.
95. Use of the IBV, H52 IBV or immunogenic composition of any one of clauses 1 to 60 for the manufacture of a medicament.
96. Use of the IBV, H52 IBV or immunogenic composition of any one of clauses 1 to 60 for the treatment and/or prophylaxis of IBV infections in a subject.
97. An immunogenic composition comprising an H52 IBV (infectious bronchitis virus) encoding for a heterologous DMV/1639 S (spike) protein, wherein said H52 IBV comprises a Nucleocapsid (N) protein, Envelope (E) protein or Membrane glycoprotein (M) having or consisting of or comprising the amino acid sequence as shown for AAK91809 (SEQ ID NO 6), AAK27163 (SEQ ID NO:7), SEQ ID NO 8, AAL26888 (SEQ ID NO:9), SEQ ID NO 10, AAK83031 (SEQ ID NO:11) or SEQ ID NO 12 or a sequence having at least 90%, 95%, 96%, 97%, 98%, 99%, 99.2%, 99.5%, 99.6%, 99.7%, 99.8%, 99.9%, 99.95%, 99.98% or 99.99% sequence identity thereto, and, wherein the heterologous DMV/1639 S protein is consisting of or comprising the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.
98. The immunogenic composition of clause 97, wherein the heterologous DMV/1639 S (spike) protein is the full length Spike protein.
99. The immunogenic composition of clause 97 or 98, wherein the heterologous DMV/1639 S (spike) protein is the Ectodomain of the spike protein.
100. The immunogenic composition of any one of clauses 97 to 99, wherein the IBV is attenuated.
101. A method of preparing an immunogenic composition for the treatment and/or prophylaxis of IBV infections in a subject comprising:
   a.) providing an H52 IBV comprising a spike (S) protein, Nucleocapsid (N) protein, Envelope (E) protein or Membrane glycoprotein (M) having or consisting of or comprising the amino acid sequence as shown for AAK27168 (SEQ ID NO:4), SEQ ID NO:5, AAK91809 (SEQ ID NO:6), AAK27163 (SEQ ID NO:7), SEQ ID NO:8, AAL26888 (SEQ ID NO:9), SEQ ID NO:10, AAK83031 (SEQ ID NO:11) or SEQ ID NO:12 or a sequence having at least 90%, 95%, 96%, 97%, 98%, 99%, 99.2%, 99.5%, 99.6%, 99.7%, 99.8%, 99.9%, 99.95%, 99.98% or 99.99% sequence identity thereto; and
   b.) providing a heterologous DMV/1639 S protein having or consisting of or comprising the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.
   c.) replacing the spike protein of H52 IBV of a) with said heterologous DMV/1639 S (spike) protein of b) to have an H52 IBV with a heterologous DMV/1639 S protein; and
   d.) obtaining said H52 IBV with a heterologous DMV/1639 S protein; and
   e.) addition of a pharmaceutically acceptable carrier.
102. The method of clause 101, wherein the heterologous DMV/1639 S (spike) protein is the full length Spike protein.
103. The method of clause 101, wherein the heterologous DMV/1639 S (spike) protein is the Ectodomain of the Spike protein.
104. The method of any one of clauses 101 or 103, wherein said pharmaceutically acceptable carrier is selected from the group consisting of solvents, dispersion media, coatings, stabilizing agents, diluents, preservatives, antibacterial and antifungal agents, isotonic agents, adsorption delaying agents, adjuvants, immune stimulants, and combinations thereof.
105. The method of any one of clauses 101 or 104, wherein the IBV is attenuated.
106. The IBV, H52 IBV or immunogenic composition of any one of clauses 1 to 60 for use as a coarse spray vaccine or for use in coarse spray application.

### Sequences Overview

SEQ ID NO:1: full length spike (S) protein of DMV/1639 with L270C mutation.
SEQ ID NO:2: ectodomain of the spike (S) protein of DMV/1639 with L270C mutation.
SEQ ID NO:3: EU817497 (H52 IBV nucleotide sequence).
SEQ ID NO:4: AAK27168 (H52 IBV spike (S) protein amino acid sequence).
SEQ ID NO:5: H52 IBV spike (S) protein.
SEQ ID NO:6: AAK91809 (H52 IBV nucleocapsid (N) protein amino acid sequence).
SEQ ID NO:7: AAK27163 (H52 IBV nucleocapsid (N) protein amino acid sequence).
SEQ ID NO:8: H52 IBV nucleocapsid (N) protein.
SEQ ID NO:9: AAL26888 (H52 IBV envelope (E) protein amino acid sequence).
SEQ ID NO:10: H52 IBV envelope (E) protein.
SEQ ID NO:11: AAK83031 (H52 IBV membrane glycoprotein (M) protein amino acid sequence).
SEQ ID NO:12: H52 IBV membrane glycoprotein (M) protein.
SEQ ID NO:13: pUC57-s IBV-5-1b-S-SIR-3T donor plasmid (H52 rIBV donor sequence)
SEQ ID NO:14: pUC57-Brick H52 rIBV DMV S donor plasmid
SEQ ID NO:15 to SEQ ID NO:19: Primers
SEQ ID NO:20: full length spike (S) protein of DMV/1639 and QCX19619
SEQ ID NO:21: ectodomain of the spike (S) protein of DMV/1639
SEQ ID NO: 22: QGM12378
SEQ ID NO: 23: QGM12391
SEQ ID NO: 24: QGM12404
SEQ ID NO: 25: QGM12416
SEQ ID NO: 26: QGM12429
SEQ ID NO: 27: QRG28806
SEQ ID NO: 28: QRG28819
SEQ ID NO: 29: UXN85484
SEQ ID NO: 30: UXN85494
SEQ ID NO: 31: UXN85504
SEQ ID NO: 32: UZP65106

### EXAMPLES

The following examples are set forth below to illustrate specific embodiments of the present invention. These examples are merely illustrative and are understood not to limit the scope or the underlying principles of the present invention.

### Example 1

Generation of Recombinant IBV H52 in which the Coding Sequence for the H52 Spike is Replaced by the Coding Sequence for a Heterologous Spike for DMV

### Generation of the Donor Plasmid

The replacement of the H52 spike by the DMVspike is described in detail: The DMV spike nucleic acid coding sequence (SEQ ID NO:1) from GenBank accession MK878536 with the sequence altered to cause a L270C residue change is used as a template to replace the IBV H52 spike nucleic acid coding sequence in the IBV sequence from the pUC57-s IBV-5-1b-S-SIR-3T donor plasmid described by van Beurden et al 2017 (Virol J 14, 109), hereafter referred to as H52 rIBV donor sequence (SEQ ID NO:13). Bases 1,220 to 4,708 of SEQ ID NO:13 (H52 rIBV donor sequence) are replaced with the IBV DMV spike nucleic acid coding sequence (SEQ ID NO:1). The IBV sequence is synthesized and inserted into the vector pUC57-Brick by a commercial supplier to generate the pUC57-Brick H52 rIBV DMV S donor plasmid (SEQ ID NO:14) in which the DMV spike is encoded by bases 3,741 to 7,241.

### Targeted RNA Recombination and Rescue of Recombinant IBV

For the generation of recombinant IBV the method of targeted RNA recombination based on that described by van Beurden et al 2017 (Virol J 14, 109) is applied. In brief, the H52 murinized (m)IBV is generated as described. For the generation of H52 rIBV DMV S, LR7 cells are infected with H52 mIBV and electroporated with in vitro transcript generated from the pUC57-Brick H52 rIBV DMV S donor plasmid and subsequently injected into 9-day-old embryonated SPF chicken eggs (Charles River). After 7 days of incubation at 37° C., the allantoic fluid of all eggs are analyzed separately for the rescue of recombinant IBV after RNA isolation by the MagMAX™-96 Viral RNA Isolation Kit (Applied Biosystems™) and by using the Superscript™ IV One-Step RT-PCR System (Invitrogen™). Primers IBV.M41.F12.4 and DMV GA9977 1836R binding in H52 IBV lab and DMV IBV S are used (Table 1) which are specific for recombinant IBV but not mIBV. Positive allantoic fluid from one egg is selected for expansion in 11-day-old SPF embryonated eggs followed by one round of end-point dilution in 9-day-old SPF eggs. RNA isolation from limiting dilution samples is performed using the MagMAX™-96 Viral RNA Isolation Kit (Applied Biosystems™) with the KingFisher™ Flex Purification System (Thermo Scientific™). RNA is subsequently analyzed for the presence of rIBV by RT-qPCR primers IBV 5′GU391 and IBV 5′GL533 with probe IBV 5′G Probe labeled with FAM dye and Iowa Black quencher (Table 1) as described by Callison et al 2006 (J Virol Methods 138, 60-65). Allantoic fluid from an egg with high rIBV viral load inoculated with the highest dilution is used for rIBV propagation in 9-to-11-day-old embryonated SPF chicken eggs. For production of a vaccine stock to be used in efficacy studies, the rIBV is passaged up to pass 10 in SPF embryonated eggs.

TABLE 1
Primers to identify recombinant IBV after rescue

| Name | Sequence |
|---|---|
| IBV.M41.F12.4 | TGACTTGGTTTGAAGATGGC |
| | (SEQ ID NO: 15) |
| DMV GA9977 1836R | AGGGCTGGTTTGCTCAGAC |
| | (SEQ ID NO: 16) |
| IBV 5′GU391 | GCTTTTGAGCCTAGCGTT |
| | (SEQ ID NO: 17) |
| IBV 5′GL533 | GCCATGTTGTCACTGTCTATTG |
| | (SEQ ID NO: 18) |
| IBV 5′G Probe | CACCACCAGAACCTGTCACCTC |
| | (SEQ ID NO: 19) |

Similarly, further donor constructs are generated using the DMV spike nucleic acid coding sequences of SEQ ID NO:2 (only ectodomain of SEQ ID NO:1), SEQ ID NO:20 (similar to SEQ ID NO:1, but no single amino acid mutation) and SEQ ID NO:21 (only ectodomain of SEQ ID NO:1, but no single amino acid mutation).

### Example 2

### Preparation of Vaccine and Challenge Virus

To demonstrate the efficacy of the H52 rIBV with heterologous DMV spike in chickens, an aliquot of the virus vaccine stock is thawed and 10-fold diluted in Vegitone to determine the 50% embryo infectious dose (EID50) by inoculation of 0.2 ml into 5 9-to-11 day old embryonated chicken eggs per dilution. Eggs are incubated at 37° C. for 7 days. Embryo death occurring 24 hours post-inoculation is excluded from the titration. 7 days post-inoculation, the infectivity ratio for each dilution is counted by totaling the number of dead embryos and embryos showing IBV-specific lesions such as stunting, curling, and clubbed down. The EID₅₀ is calculated using the method of Reed and Muench 1938 (Am J Epidemiol 27, 493-497). The DMV/1639 challenge virus is provided by Dr. Brian Jordan at the UGA Poultry Diagnostic and Research Center and is titrated in the same method.

### Example 3

### Evaluation of the Efficacy of the H52 RIBV DMV S Vaccine Against IBV DMV/1639 Challenge Virus

The efficacy of the H52 rIBV DMV S vaccine against IBV DMV/1639 challenge is evaluated as follows: On Day 0, 30 one-day-old SPF chickens (SPAFAS) are assigned to groups 1-3. Chickens in groups 2 and 3 are placed in isolation units (one unit per group, 10 chickens per unit) without receiving treatment and prior to vaccination. Each chicken from group 1 is vaccinated via the intraocular route with 0.06 ml (0.3 ml per eye) of 1×10³ EID₅₀ of H52 rIBV DMV S (SEQ ID NO:1). After vaccination, chickens in group 1 are placed into isolation units (2 units per group, 10 chickens per unit) and neck-banded for individual identification. All chickens in groups 1 and 2 are challenged on day 21 by the intraocular route with 1×10^{2.5} EID₅₀/0.06 ml of IBV DMV/1639 challenge. Throughout the study, all chickens are observed for well-being. All chickens are observed for mortality for 5 days post-challenge. On day 26 (5 days post-challenge), tracheal swabs are collected for virus isolation (VI) in SPF chicken embryos from all remaining chickens in groups 1-3. The virus isolation test is performed according to the requirements for immunogenicity testing as described by the US Title 9 Code of Federal Regulations, section 113.327 (Bronchitis Vaccine). Thereafter chickens are terminated.

Results are shown in Table 2. Chickens vaccinated with H52 rIBV DMV S (SEQ ID NO:1) and subsequently challenged with DMV/1639 (Group 1) show a low virus isolation frequency of 10% (90% protection efficacy), which meets the acceptance criteria for efficacy. Group 2, in which chickens are not vaccinated but are challenged with DMV/1639, showed 100% positivity for virus isolation. Group 3, in which chickens are not vaccinated or challenged, remains completely negative for virus isolation.

TABLE 2
Experimental results

| | | H52 rIBV DMV S | DMV/1639 | Virus isolation |
|---|---|---|---|---|
| | | (SEQ ID NO: 1) | challenge | positive tracheal |
| Group | # | (1 × 10³ | (1 × 10^{2.5} | swabs/Total (% |
| # | Chickens | EID₅₀/dose) | EID₅₀/dose) | Protection) |
| 1 | 20 | Yes | Yes | 2/20 (90%) |
| 2 | 10 | No | Yes | 10/10 (0%) |
| 3 | 10 | No | No | 0/10 (100%) |

Under the experimental conditions tested in this trial and based on virus isolation results, vaccine candidate H52 rIBV DMV S (SEQ ID NO:1) confers 90% protection against IBV DMV/1639 homologous challenge. Similar results and protection rates are found for vaccines H52 rIBV DMV S (SEQ ID NO:2), H52 rIBV DMV S (SEQ ID NO:20) and H52 rIBV DMV S (SEQ ID NO:21).
This is the first time showing that a recombinant approach using a DMV/1639 spike sequence (varying significantly from IBV spike sequences used in any other vector approach so far) protects against a DMV/1639 challenge. Further, the codified 9-CFR testing of virus isolation in embryos is used for showing high efficacy of the vaccine.

### Example 4

Evaluation of the Efficacy of the H52 RIBV DMV S Vaccine Administered Via Spray Against IBV DMV/1639 Challenge Virus

The efficacy of the H52 rIBV DMV S vaccine administered via coarse spray against IBV DMV/1639 challenge is evaluated as follows: On Day 0, 40 one-day-old SPF chickens (AVS Bio) are assigned to groups 1-4. Chickens in groups 3 (n=10) and 4 (n=5) are placed in one isolation unit each without receiving treatment and prior to vaccination of the other groups. All chickens from groups 1 and 2 (n=25 total) are placed into a single vaccination tray, confining all the birds into one quadrant of the tray to mimic bird density of a full tray. Chickens are then vaccinated by coarse spray with 1×10⁴ EID₅₀ of H52 rIBV DMV S per dose (in sterile distilled water) using the Boehringer Ingelheim's Spra-Vac®L1 cabinet, set to deliver 21 mL per tray of 100 chickens (approximately 0.21 mL/chicken). Following vaccination, the chickens remain in the vaccination tray for at least 1 hour to mimic field conditions. After 1 hour, chickens are neck-banded for individual identification and placed into one isolation unit per group. Twenty eight days after vaccination, all chickens from groups 1 and 3 are challenged by the intraocular route with 1×10^{2.5} EID₅₀/0.03 ml of IBV DMV/1639 challenge. Throughout the study, all chickens are observed for well-being. All chickens are observed for clinical signs and mortality for 5 days post-challenge. On day 33 (5 days post-challenge), tracheal swabs are collected from all remaining chickens in groups 1-4 for virus isolation (VI) in SPF embryonated chicken eggs. Chickens are euthanized after sample collection. The virus isolation test is performed according to the requirements for immunogenicity testing as described by the US Title 9 Code of Federal Regulations, section 113.327 (Bronchitis Vaccine).

Results are shown in Table 3. None of the chickens vaccinated with H52 rIBV DMV showed clinical signs post-challenge with DMV/1639 (Group 1). Chickens vaccinated with H52 rIBV DMV and subsequently challenged with DMV/1639 (Group 1) show a low virus isolation frequency of 10% (90% protection efficacy), which meets the criteria for efficacy. The non-vaccinated challenged chickens (Group 3), showed 100% positivity for virus isolation. The vaccinated/non-challenged (Group 2) and the non-vaccinated/non-challenged (Group 4) chickens, remain negative for virus isolation at day 33 (5 days post-challenge).

TABLE 3
Experimental Results

| | | | | | Virus |
|---|---|---|---|---|---|
| | | | | Number | isolation |
| | | | | of | positive |
| | | H52 | DMV/ | chickens | tracheal |
| | | rIBV | 1639 | with | swabs/ |
| | | DMV S | challenge | clinical | Total (% |
| | | (1 × 10⁴ | (1 × 10^{2.5} | signs | Protection)- |
| Group | # | EID₅₀/ | EID₅₀/ | post- | 5 days post- |
| # | Chickens | dose) | dose) | challenge | challenge |
| 1 | 20 | Yes | Yes | 0/20 | 2/20 (90%) |
| 2 | 5 | Yes | No | N/A | 0/5 |
| 3 | 10 | No | Yes | 9/10 | 10/10 (0%) |
| 4 | 5 | No | No | N/A | 0/5 |

In conclusion, based on the virus isolation results from this trial, the H52 rIBV DMV S vaccine provides 90% protection against homologous challenge with DMV/1639, when administered via coarse spray, complying with the requirements for efficacy described in the US Title 9 Code of Federal Regulations, section 113.327. Consequently, the H52 rIBV DMV S vaccine is the only vaccine described so far that complies with the requirements of US Title 9 Code of Federal Regulations and, thus, could get licensed for application via coarse spray. Since most chicken hatcheries commonly use coarse spray vaccination cabinets for mass vaccination, this is a clear advantage over the prior art vaccines.

1. An IBV (infectious bronchitis virus) encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.

2. A H52 IBV (infectious bronchitis virus) having at least 85%, 90%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 3 encoding for a heterologous IBV spike protein consisting of or comprising the amino acid sequence as shown in SEQ ID NO: 1, 2, 20 or 21 or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto or fragment thereof.

3. A H52 IBV (infectious bronchitis virus) encoding for a heterologous DMV/1639 IBV spike protein or fragment thereof.

4. An immunogenic composition comprising the IBV (infectious bronchitis virus) of claim 1.

5. The IBV of claim 1, wherein the IBV is selected from the group consisting of: H52, H120, 4/91 and QX.

6. The IBV of claim 1, wherein the heterologous IBV spike protein is a heterologous DMV/1639 IBV spike protein, and the DMV/1639 spike protein consists of or comprises a sequence selected from the list consisting of: SEQ ID NO: 20 (QCX19619), SEQ ID NO: 22 (QGM12378), SEQ ID NO: 23 (QGM12391), SEQ ID NO: 24 (QGM12404), SEQ ID NO: 25 (QGM12416), SEQ ID NO: 26 (QGM12429), SEQ ID NO: 27 (QRG28806), SEQ ID NO: 28 (QRG28819), SEQ ID NO: 29 (UXN85484), SEQ ID NO: 30 (UXN85494), SEQ ID NO: 31 (UXN85504) and SEQ ID NO: 32 (UZP65106) or a sequence having at least 85%, 90%, 95%, 96%, 97%, 98%, 99%, 99.2%, 99.5%, 99.6%, 99.7%, 99.8%, 99.9%, 99.95%, 99.98% or 99.99% sequence identity thereto.

7. The IBV of claim 1, wherein the heterologous IBV spike protein is a heterologous DMV/1639 IBV spike protein, and the DMV/1639 spike protein consists of or Response to Notice to Incomplete Reply mailed Oct. 30, 2024 and Notice to File Missing Part mailed Aug. 19, 2024 comprises the sequence as shown in SEQ ID NO: 20 (QCX19619) or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.

8. The IBV of claim 1, wherein the heterologous IBV spike protein is a heterologous DMV/1639 IBV spike protein, and the heterologous DMV/1639 spike protein has at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity to SEQ ID NO: 1, 2, 20 or 21 or the heterologous DMV/1639 spike protein consists of or comprises the amino acid sequence as shown in SEQ ID NO: 1, 2, 20 or 21 or a sequence having at least 85%, 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto.

9. The IBV of claim 1, wherein the heterologous IBV spike protein is a heterologous DMV/1639 IBV spike protein, and the heterologous DMV/1639 spike protein has one or more amino acid(s) selected from the list consisting of: 242T, 489F and 1038L.

10. The IBV of claim 1, wherein the heterologous IBV spike protein is a heterologous DMV/1639 IBV spike protein, and the heterologous DMV/1639 spike protein has the amino acid Cysteine at amino acid position 270 or wherein the heterologous DMV/1639 spike protein has the amino acid 270C.

11. The IBV of claim 9, wherein the numbering of amino acid positions refers to the amino acid positions in the spike protein as given in SEQ ID NO:1.

12. The IBV of claim 1, wherein the fragment of the heterologous DMV/1639 spike protein has a length of at least 1000, at least 1050 or at least 1077 amino acids.

13. The IBV of claim 1, wherein the IBV is attenuated or the IBV is a recombinant IBV.

14. The immunogenic composition of claim 4, wherein the immunogenic composition is a vaccine.

15. A kit comprising the IBV of claim 1.

16. A method for immunizing a subject comprising administering to such subject the immunogenic composition according to claim 4.

17. A method of treating or preventing clinical signs caused by IBV in a subject, the method comprising administering to the subject a therapeutically effective amount of the immunogenic composition according to claim 4.

18. A method of reducing viral load in a subject, in comparison to a subject of a non-immunized control group of the same species, the method comprising administering to the subject a therapeutically effective amount of the immunogenic composition according to claim 4.

19. The method of claim 16, wherein the subject is poultry and/or the immunogenic composition is administered via coarse spray.

20. The IBV of claim 1 for use as a coarse spray vaccine or for use in coarse spray application.
